(19) United States
(12) Patent Application Publication
(10) Pub. No.: US 2014/0148400 A1
(43) Pub. Date: May 29, 2014

(54) CARBONIC ANHYDRASE INHIBITORS WITH ANTIMETASTATIC ACTIVITY

(71) Applicants: Claudiu Supuran, Firenze (IT); Shoukat Dedhar, Richmond (CA); Fabrizio Carta, Irgoli (IT); Jean-Yves Winum, St. Andre de Sangonis (FR); Paul C. McDonald, Coquitlam (CA)

(72) Inventors: Claudiu Supuran, Firenze (IT); Shoukat Dedhar, Richmond (CA); Fabrizio Carta, Irgoli (IT); Jean-Yves Winum, St. Andre de Sangonis (FR); Paul C. McDonald, Coquitlam (CA)

(73) Assignees: METASIGNAL THERAPEUTICS INC., Richmond (CA); CNRS-DIRE, Paris (FR); UNIVERSITE MONTPELLIER 2 SCIENCES ET TECHNIQUES, Montpellier (FR)

(21) Appl. No.: 14/078,455

(22) Filed: Nov. 12, 2013

Related U.S. Application Data
(63) Continuation of application No. 13/989,699, now abandoned.
(60) Provisional application No. 61/417,449, filed on Nov. 28, 2010.

(51) Int. Cl. C07D 311/20; A61K 31/7048; C07D 405/12; A61K 45/06; A61K 31/37; C07D 405/14; A61K 31/506; C07H 17/075; A61K 31/4192

(52) U.S. Cl. CPC C07D 311/20; C07H 17/075; A61K 31/7048; C07D 405/12; A61K 31/4192; A61K 31/37; C07D 405/14; A61K 31/506; A61K 45/06
USPC 514/27; 536/18.1; 548/256; 514/359; 549/289; 514/457; 549/288; 544/310; 514/274

(57) ABSTRACT

Derivatized coumarin-based pharmaceutical compositions and methods to use them are provided. The compositions are characterized in that they inhibit the activity of tumor-related CAIX and CAXII to a greater degree than they inhibit the activity of CAI and CAII. The compositions can be used to suppress tumor growth and/or suppress tumor metastases in a mammal.

### BACKGROUND OF THE INVENTION

1. Field of Invention

The invention is in the field of novel coumarins, thiocoumarins, and glycosylated coumarins, and their use as inhibitors of carbonic anhydrase IX and XII, in the treatment of hypoxic and metastatic cancer.

2. Description of Related Art

Carbonic anhydrases are involved in numerous physiological and pathological processes in mammals, including respiration and transport of CO₂/bicarbonate between metabolizing tissues and lungs, pH and CO₂ homeostasis, electrolyte secretion in a variety of tissues/organs, biosynthetic reactions (e.g., gluconeogenesis, lipogenesis and ureagenesis), bone resorption, calcification, tumorigenicity, and many other physiological and pathological processes studied in humans, as well as the growth and virulence of various fungal/bacterial pathogens.^{2,4-11} In addition to the established role of CA inhibitors (CAIs) as diuretics and antiglaucoma drugs, it has recently emerged that they have potential as anticonvulsant, antiobesity, anticancer and antiinfective drugs.^{2,4-11} Many of the mammalian CA isozymes involved in these processes are important therapeutic targets with the potential to be inhibited or activated to treat a wide range of disorders.^{2,4} However a critical barrier to the design of CAIs as therapeutic agents is related to the high number of isoforms in humans, their rather diffuse localization in many tissues/organs, and the lack of isozyme selectivity of the presently available inhibitors of the sulfonamide/sulfamate type.^{2,4}

The CA family of enzymes is widespread all over the phylogenetic tree (with 16 different α-CA isozymes presently known in mammals), and is inhibited by compounds which bind to the catalytically critical Zn(II) ion from the enzyme active site (or the water/hydroxide ion coordinated to it): the sulfonamides, their bioisosteres (sulfamates, sulfamides, N-substituted sulfonamides, etc), some metal complexing anions, and (thio)phenols among others.^{2,4} The coumarins, such as the natural product coumarin 1 for which this effect was initially reported,^{1,3} do not have any obvious functionality to confer them potent CA inhibitory activity.

Coumarin 6-(1S-hydroxy-3-methylbutyl)-7-methoxy-2H-chromen-2-one and the simple, unsubstituted coumarin (see structures 1 and 2) were nonselective, potent inhibitors against all investigated human CA isoforms.

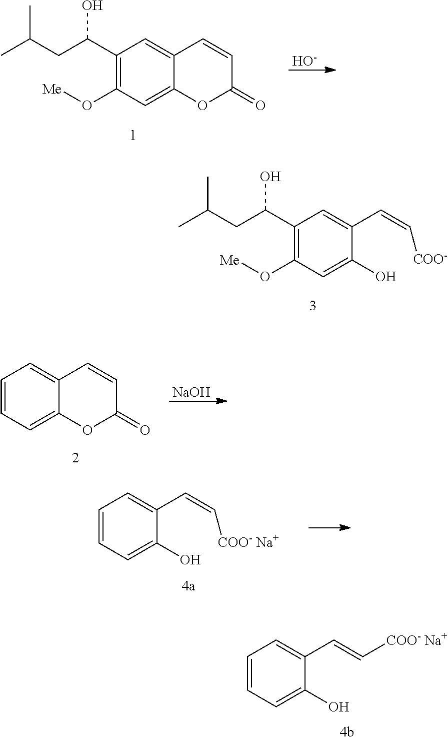

Other semisynthetic coumarin compounds have been shown to inhibit the metalloenzyme carbonic anhydrases (Maresca, A; Temperini, C; Vu, H et al. J. Am. Chem. Soc. 2009, 131, 3057-3062; Maresca, A; Temperini, C; Pochet, L. et al. J. Med. Chem. 2010, 53, 335-344; Maresca, A; Supuran, C. Bioorganic & Medicinal Chemistry Letters 2010, 20, 4511-4514).

Certain “novobiocin” compounds are disclosed in U.S. Pat. No. 7,608,594 by Blagg et al.

### SUMMARY OF THE INVENTION

According to the invention, coumarin and thiocoumarin compositions suitable for the treatment of hypoxic or metastatic cancer are provided, having the general structures I to VI:

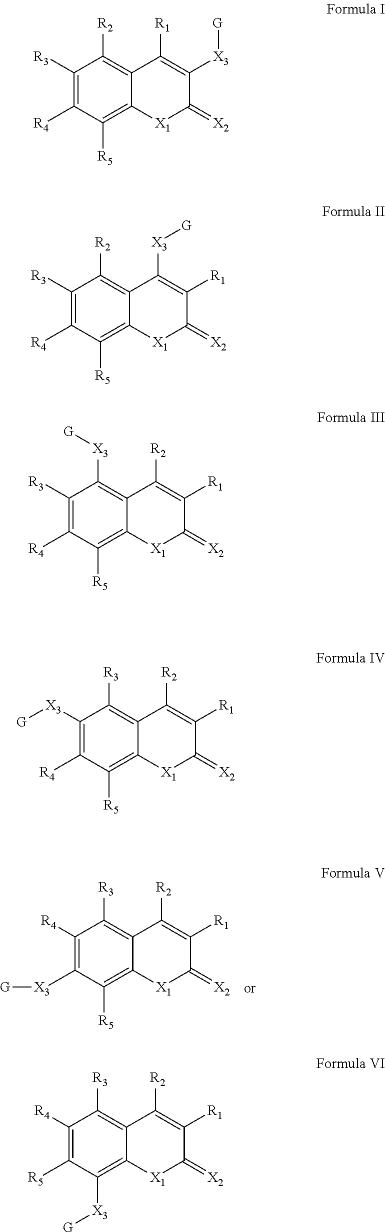

capable of inhibiting the activity of tumor-related CAIX and CAXII to a greater degree than they inhibit the activity of CAI and CAII in vitro, and a pharmaceutically acceptable excipient, are provided.

Also provided for the treatment of hypoxic metastatic cancer, and capable of inhibiting CAIX and CAII to a greater degree than they inhibit the activity of CAI and CAII as measured in vitro, are the active metabolites of the pharmaceutical compounds of the invention, namely 2-hydroxycinnamic acids and 2-hydroxy-thiocinnamic acid derivatives having the general structures VII-XII.

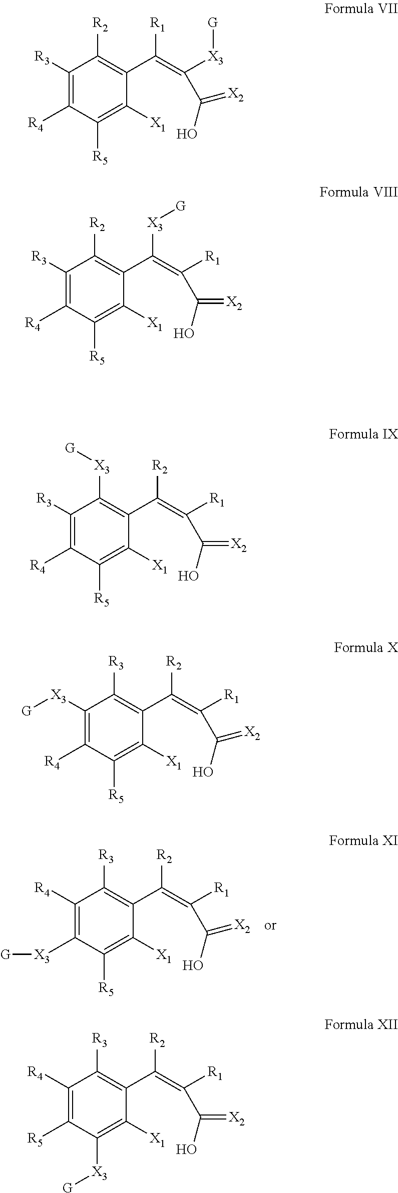

and a pharmaceutically acceptable excipient.

According to one aspect of the invention, there are provided glycosylated coumarins capable of inhibiting the activity of tumor-related CAIX and CAXII to a greater degree than they inhibit the activity of CAI and CAII in vitro, according to the above Formulae, wherein G is a glycosyl group, or a heterocyclic sugar according to the following general schema:

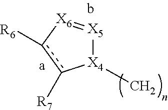

Wherein for formulae I, II, III, IV, V, VI, VII, VIII, IX, X, XI, XII:
a=-single bond-, -double bond-;
b=-single bond-, -double bond-;

### X₁, X₂═O,S;

X₃═—O—, —NH—, —S—, -single bond-;

### X₄═—N—,—C—;

### X₅, X₆═—N—, —C—, —O—;

n=0,1.

R₁═H; and R₂; R₃; R₄; R₅; R₆ and R₇ are independently ═H, alkyl, alkenyl, alkynyl, cycloalkyl, heterocyclic, aryl, or halogen atom.

In another aspect of the invention, there are provided non-glycosylated coumarins for use as pharmaceuticals or as intermediates in the synthesis of glycosylated coumarin synthesis. In these non-glycosylated coumarins, G is H, alkyl, alkenyl, alkynyl, cycloalkyl, heterocyclic, or aryl.

According to an aspect of the invention, there are provided compositions suitable for the treatment of hypoxic or metastatic cancer and capable of inhibiting the activity of tumor-related CAIX and CAXII to a greater degree than they inhibit the activity of CAI and CAII in vitro, comprising any one of:

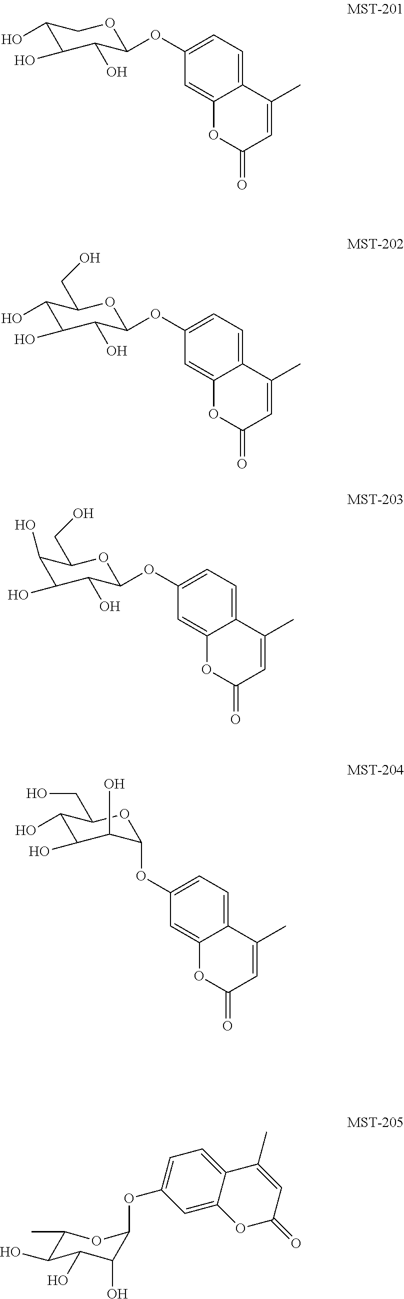 [SMILES: Cc1cc(=O)oc2cc(OC3OCC(O)C(O)C3O)ccc12 ; Cc1cc(=O)oc2cc(OC3OC(CO)C(O)C(O)C3O)ccc12 ; Cc1cc(=O)oc2cc(OC3OC(CO)C(O)C(O)C3O)ccc12 ; Cc1cc(=O)oc2cc(OC3OC(CO)C(O)C(O)C3O)ccc12 ; Cc1cc(=O)oc2cc(OC3OC(C)C(O)C(O)C3O)ccc12]

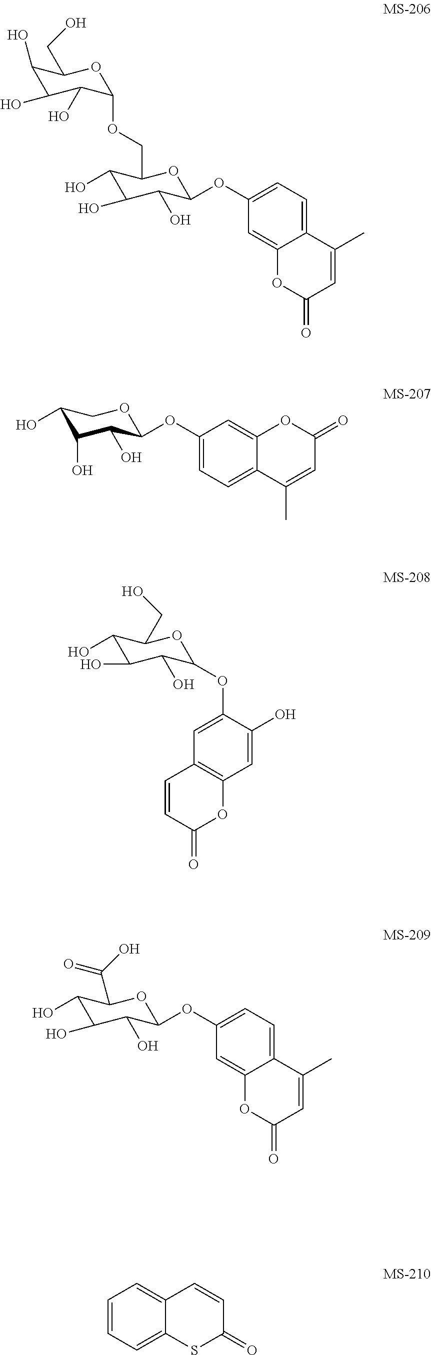

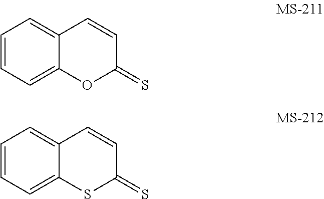

and a pharmaceutical excipient.

According to some aspects of the invention, for general structure Formula V above, G may be (CH₂)ₙG1, and

X1=X2=O, X₃═O, n=2, G1=1-(2′,4′,6′-trimethylpyridino) (Compound MST-213); or
X1=X2=O, X₃═O, n=3, G1=Co₂(CO)₆(Acetylene) (Compound MST-214); or
where X₁═O, X₂═S (Compound MST-216) and where X1=X2=O, X₃═O, n=1, G1=Co₂(CO)₆(Acetylene) (Compound MST-224); or where
X1=X2=O, X₃═NH, R2=methyl, n=0, G1=4-methylbenzenesulfonyl (Compound MST-215); or where
X1=O, X2=S, X₃═O, n=1, G1=acetylene (Compound MST-217); or where
X1=O, X₂═S, X₃═O, n=1, G1=vinyl (Compound MST-218); or where
X1=X2=O, X₃═O, n=2, G1=BOC-amino (Compound MST-219); or where
X1=X2=O, X₃═O, n=1, G1=1-[2-(5-methylpyrimidine-2,4-dione-1-yl)-5-(hydroxymethyl)-tetrahydrofuran-3-yl]-1,2,3-triazol-4-yl (Compound MST-221);
or where X1=X2=O, X₃═O, n=1, G1=1,2,3-triazol-4-yl (Compound MST-223); or where
X1=X2=O, X₃═O, n=1, G1=1-(2′-chlorophenyl)-1,2,3-triazol-4-yl, 1-(2′-bromophenyl)-1,2,3-triazol-4-yl, 1-(2′-fluorophenyl)-1,2,3-triazol-4-yl, and 1-(2′-iodophenyl)-1,2,3-triazol-4-yl (Compounds MST-225, MST-226, MST-229 and MST-227, respectively).

For other non glycosylated coumarin compounds of the invention, there are provided a compositions for the treatment of hypoxic metastatic cancer and capable of inhibiting the activity of CAIX and CAXII to a greater degree than they inhibit the activity of CAI and CAII, where a general structure according to Formula V is substituted such that X1=X2=O, X3 is a single bond, R2=methyl, G is 1-(2′,4′,6′-trimethylpyridino)-(Compound MST-220); or where G is 4-((2-oxo-2H-chromen-7-yloxy)methyl)-1,2,3-triazol-1-yl (Compound MST-222).

Further compositions provided are capable of inhibiting the activity of tumor-related CAIX and CAXII to a greater degree than they inhibit the activity of CAI and CAII in vitro, for uses according to the invention include:

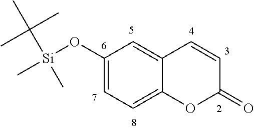 [SMILES: CC(C)(C)[Si](C)(C)Oc1ccc2oc(=O)ccc2c1]

6-(tert-Butyldimethylsilyloxy)-2H-chromen-2-one (MST-230);

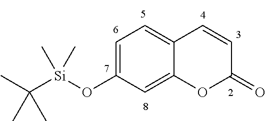 [SMILES: CC(C)(C)[Si](C)(C)Oc1ccc2ccc(=O)oc2c1]

7-(tert-Butyldimethylsilyloxy)-2H-chromen-2-one (MST-231);

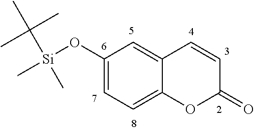 [SMILES: CC(C)(C)[Si](C)(C)Oc1ccc2oc(=O)ccc2c1]

6-(tert-Butyldimethylsilyloxy)-2H-chromene-2-thione (MST-232);

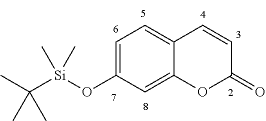 [SMILES: CC(C)(C)[Si](C)(C)Oc1ccc2ccc(=O)oc2c1]

7-(tert-Butyldimethylsilyloxy)-2H-chromene-2-thione (MST-234);

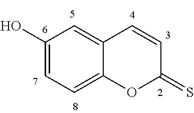 [SMILES: Oc1ccc2oc(=S)ccc2c1]

6-Hydroxy-2H-chromene-2-thione (MST-233);

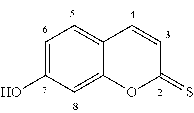 [SMILES: Oc1ccc2ccc(=S)oc2c1]

6-Hydroxy-2H-chromene-2-thione (MST-235);

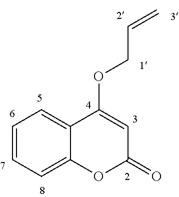

4-(Allyloxy)-2H-chromen-2-one (MST-236);

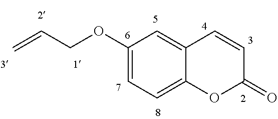

6-(Allyloxy)-2H-chromen-2-one (MST-237);

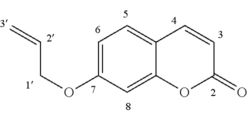

7-(Allyloxy)-2H-chromen-2-one (MST-238);

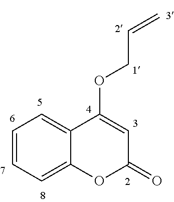

4-(Allyloxy)-2H-chromene-2-thione (MST-239);

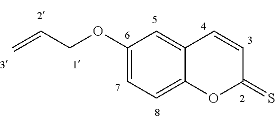

6-(Allyloxy)-2H-chromene-2-thione (MST-240);

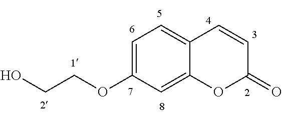 [SMILES: O=c1ccc2ccc(OCCO)cc2o1]

7-(2′-hydroxyethoxy)-2H-chromen-2-one (MST-241);

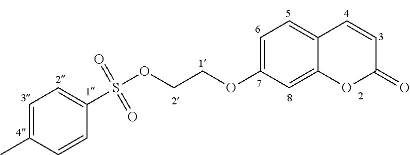 [SMILES: Cc1ccc(S(=O)(=O)OCCOc2ccc3ccc(=O)oc3c2)cc1]

2′-(2-Oxo-2H-chromen-7-yloxy)ethyl 4″-methylbenzenesulfonate (MST-242);

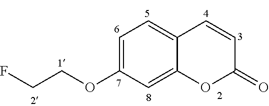 [SMILES: O=c1ccc2ccc(OCCF)cc2o1]

7-(2′-Fluoroethoxy)-2H-chromen-2-one (MST-243);

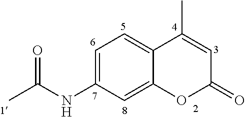 [SMILES: CC(=O)Nc1ccc2c(C)cc(=O)oc2c1]

N-(4-Methyl-2-oxo-2H-chromen-7-yl)acetamide (MST-244);

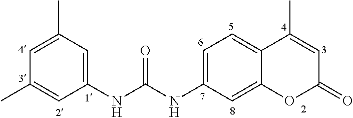 [SMILES: Cc1cc(C)cc(NC(=O)Nc2ccc3c(C)cc(=O)oc3c2)c1]

1-(3′,5′-dimethylphenyl)-3-(4-methyl-2-oxo-2H-chromen-7-yl)urea (MST-245);

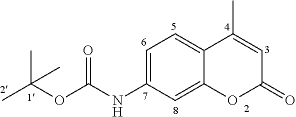 [SMILES: Cc1cc(=O)oc2cc(NC(=O)OC(C)(C)C)ccc12]

tert-Butyl 4-methyl-2-oxo-2H-chromen-7-ylcarbamate (MST-246);

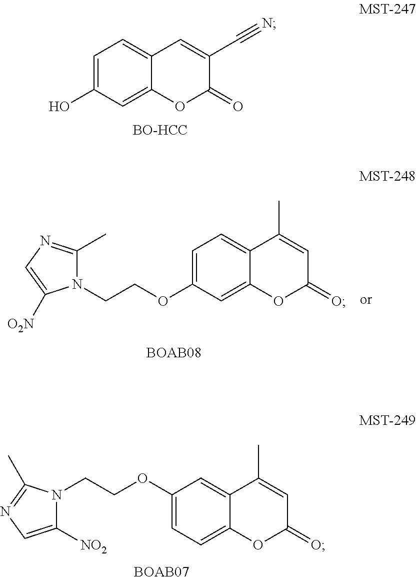

or any one or more of these combined with a pharmaceutically acceptable excipient.

There is further provided a method of suppressing tumor growth and/or suppressing tumor metastases in a mammal by treating said mammal with the compositions.

Additional anticancer agents, including other compositions of the invention, may be used in before, after or during treatment with the compositions of the invention. The treated tumors may express or overexpress CAIX and/or CAXII.

Treatable cancers and tumors include breast carcinoma, lung carcinoma, pancreatic carcinoma, renal carcinoma, ovarian cancer, prostate cancer, cervical cancer, glioblastoma, and colorectal cancer. Mammals suitable for treatment include humans.

According to another aspect of the invention, there is provided a composition for use in the manufacture of a medicament.

According to yet another aspect of the invention, there is provided the use of a composition for the preparation of a medicament for use in the treatment of hypoxic or metastatic cancer.

There is also provided a method of treating metastatic or hypoxic cancer with MST-204 or 4-methylumbellifer-7-yl-α-D-mannopyranoside and MST-205 4-methylumbellifer-7-yl- -L-rhamnopyranoside, particularly in a pharmaceutical formulation.

Also provided are methods of preparing medicaments comprising the compositions provided.

Other aspects and features of the present invention will become apparent to those ordinarily skilled in the art upon review of the following description of specific embodiments of the invention in conjunction with the accompanying figures, tables, formulae and examples.

### BRIEF DESCRIPTION OF THE DRAWINGS

In figures which illustrate embodiments of the invention,

FIG. 1A shows the chemical structure of CAIX inhibitor MST-204;

FIG. 1B shows representative bioluminescent images of metastases established following intravenous injection 4T1 cells and treatment with MST-204;

FIG. 1C shows an image of the results of quantification of tumor-derived bioluminescence shown in FIG. 1B;

FIG. 2A shows the chemical structure of CAIX inhibitor MST-205;

FIG. 2B shows representative bioluminescent images of metastases established following intravenous injection 4T1 cells and treatment with MST-205;

FIG. 2C shows a graphical representation of the quantification of the tumor-derived bioluminescence illustrated in FIG. 2B; and

FIG. 3 is a graphical representation of the data obtained with MST-205 in vivo, namely measurements of the ability of MST-205 to attenuate the growth of 4T1 primary tumors.

### DETAILED DESCRIPTION

Compositions, methods to prepare them, and methods to use them are provided in accordance with the invention. To clarify terminology used herein, compound designation MST-204 represents 4-methylumbellifer-7-yl-α-D-mannopyranoside. MST-205 represents 4-methylumbellifer-7-yl- -L-rhamnopyranoside. “Glycosylated coumarins” as used herein describes many of the group of compounds provided by the invention, namely a coumarin linked by an oxygen or sulfur to a monosaccharide such as allose, altrose, glucose, mannose, idose, galactose, talose, gulose, fructose, tagatose, sorvose, psicose, ribulose, xylulose, ribose, arabinose, xylose, lyxose, or deoxyribose.

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

“Alkyl” is a monovalent, saturated or unsaturated, straight, branched or cyclic, aliphatic (i.e., not aromatic) hydrocarbon group. In various embodiments, the alkyl group has 1-20 carbon atoms, i.e., is a C1-C20 (or C₁-C₂₀) group, or is a C1-C18 group, a C1-C12 group, a C1-C6 group, or a C1-C4 group. Independently, in various embodiments, the alkyl group has: zero branches (i.e., is a straight chain), one branch, two branches, or more than two branches; is saturated; is unsaturated (where an unsaturated alkyl group may have one double bond, two double bonds, more than two double bonds, and/or one triple bond, two triple bonds, or more than three triple bonds); is, or includes, a cyclic structure; or is acyclic. Exemplary alkyl groups include C₁ alkyl (i.e., —CH₃ (methyl)), C₂ alkyl (i.e., —CH₂CH₃ (ethyl)) and C₃ alkyl (i.e., —CH₂CH₂CH₃ (n-propyl), —CH(CH₃)₂ (i-propyl) and —CH(CH₂)₂(cyclopropyl)).

“Alkenyl” is a specie of alkyl group, where an alkenyl group has at least one carbon-carbon double bond. Exemplary alkyl groups include C₂ alkenyl (i.e., —CH═CH₂ (ethenyl)) and C₃ alkenyl (i.e., —CH═CH—CH₃ (1-propenyl), —CH₂—CH═CH₂ (2-propenyl), and —C(CH₃)═CH₂ (1-methylethenyl)).

“Alkynyl” is a specie of alkyl group, where an alkynyl group has a least one carbon-carbon triple bond. Exemplary alkyl groups include —C≡CH (ethynyl)) and —C≡C—CH₃ (1-propynyl), and —CH₂—C≡CH (2-propynyl)).

“Cycloalkyl” indicates a carbocyclic aryl group selected from phenyl, substituted phenyl, naphthyl, and substituted naphthyl. Suitable substituents on a phenyl or naphthyl ring include C₁-C₆ alkyl, C₁-C₆ alkoxy, carboxyl, carbonyl(C₁-C₆)alkoxy, halogen, hydroxyl, nitro, —SO₃H, and amino. Cycloalkyl can include “Arylenes” which are polyvalent, aromatic hydrocarbons, ring system. The ring system may be monocyclic or fused polycyclic (e.g., bicyclic, tricyclic, etc.). In various embodiments, the monocyclic arylene group is C5-C10, or C5-C7, or C5-C6, where these carbon numbers refer to the number of carbon atoms that form the ring system. The arylene group may be divalent, i.e., it has two open sites that each bond to another group

“Aryl” is a monovalent, aromatic, hydrocarbon, ring system. The ring system may be monocyclic or fused polycyclic (e.g., bicyclic, tricyclic, etc.). In various embodiments, the monocyclic aryl ring is C5-C10, or C5-C7, or C5-C6, where these carbon numbers refer to the number of carbon atoms that form the ring system. A C6 ring system, i.e., a phenyl ring, is a preferred aryl group. In various embodiments, the polycyclic ring is a bicyclic aryl group, where preferred bicyclic aryl groups are C8-C12, or C9-C10. A naphthyl ring, which has 10 carbon atoms, is a preferred polycyclic aryl group.

“Heteroalkyl” is an alkyl group (as defined herein) wherein at least one of the carbon atoms is replaced with a heteroatom. Preferred heteroatoms are nitrogen, oxygen, sulfur, and halogen. A heteroatom may, but typically does not, have the same number of valence sites as carbon. Accordingly, when a carbon is replaced with a heteroatom, the number of hydrogens bonded to the heteroatom may need to be increased or decreased to match the number of valence sites of the heteroatom. For instance, if carbon (valence of four) is replaced with nitrogen (valence of three), then one of the hydrogens formerly attached to the replaced carbon must be deleted. Likewise, if carbon is replaced with halogen (valence of one), then three (i.e., all) of the hydrogens formerly bonded to the replaced carbon must be deleted. As another example, trifluoromethyl is a heteroalkyl group wherein the three methyl groups of a t-butyl group are replaced by fluorine.

“Heteroatom” is a halogen, nitrogen, oxygen, silicon or sulfur atom. Groups containing more than one heteroatom may contain different heteroatoms.

A sugar may be a monosaccharide or a disaccharide. Monosaccharides are the simplest carbohydrates in that they cannot be hydrolyzed to smaller carbohydrates. They are aldehydes or ketones with two or more hydroxyl groups. The general chemical formula of a monosaccharide is (C.H2O)n, with n≧3. Examples of monosaccharides include glucose (an aldohexose), fructose (ketohexose), and ribose (an aldopentose).

Each carbon atom bearing a hydroxyl group (—OH), with the exception of the first and last carbons, are asymmetric, making them stereocenters with two possible configurations each (R or S). Because of this asymmetry, a number of isomers may exist for any given monosaccharide formula. The assignment of D or L is made according to the orientation of the asymmetric carbon furthest from the carbonyl group: in a standard Fischer projection if the hydroxyl group is on the right the molecule is a D sugar, otherwise it is an L sugar.

Glucose can exist in both a straight-chain and ring form. The aldehyde or ketone group of a straight-chain monosaccharide will react reversibly with a hydroxyl group on a different carbon atom to form a heterocyclic ring with an oxygen bridge between two carbon atoms. Rings with five and six atoms are called furanose and pyranose forms, respectively, and exist in equilibrium with the straight-chain form.

“Azido sugars” are sugars are sugars wherein an hydroxy grouup has been replaced by an azido, or N³ group.

When X═O the main categories involved are:

Allose, altrose, glucose, mannose, idose, galactose, talose, gulose, fructose, tagatose, sorbose, psicose, ribulose, Xylulose, ribose, arabinose, xylose, lyxose, and deoxyribose.

### Synthesis

### Selected Chemical Routes Involving Azidosugars:

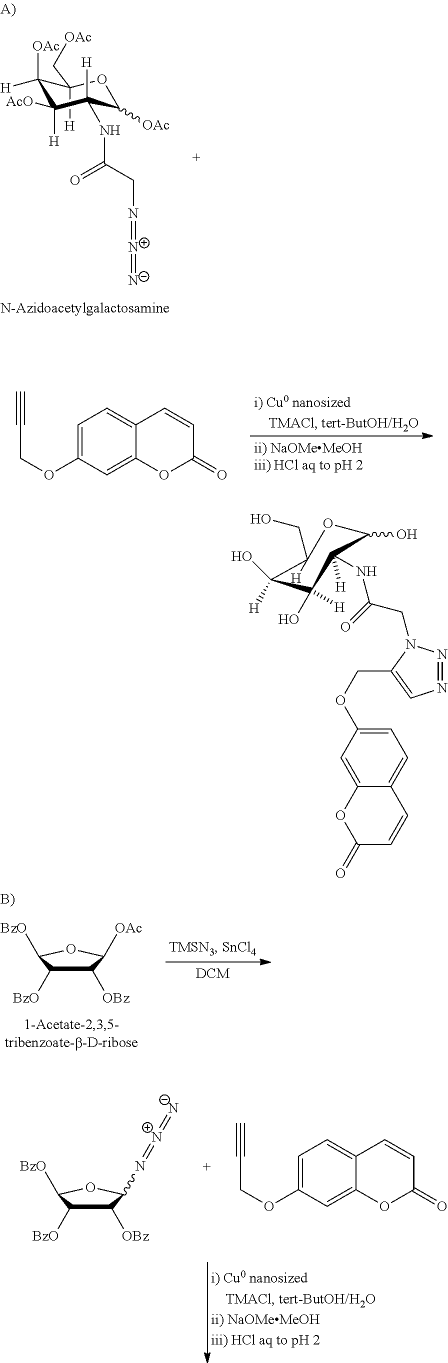

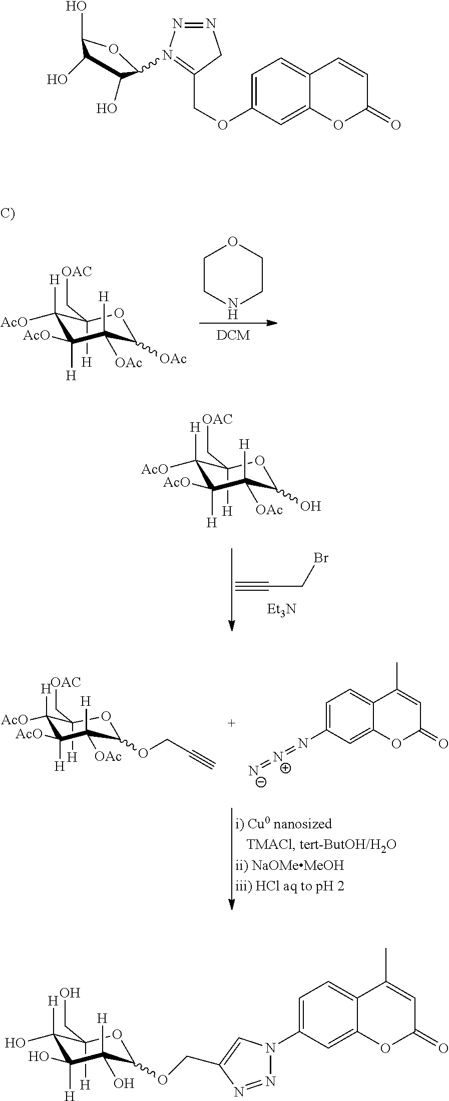

As used herein, and unless otherwise specified, the term heterocyclic encompasses both substituted and unsubstituted carbocyclic and heterocyclic groups. In one embodiment, the substitution present on a carbocyclic or heterocyclic group is selected from alkyl, heteroalkyl, aryl, and heteroaryl, preferably alkyl and heteroalkyl.

“Pharmaceutically acceptable salt” and “salts thereof” in the compounds of the present invention refers to acid addition salts and base addition salts.

Acid addition salts refer to those salts formed from compounds of the present invention and inorganic acids such as hydrochloric acid, hydrobromic acid, sulfuric acid, nitric acid, phosphoric acid and the like, and/or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Base addition salts refer to those salts formed from compounds of the present invention and inorganic bases such as sodium, potassium, lithium, ammonium, calcium, magnesium, iron, zinc, copper, manganese, aluminum salts and the like. Suitable salts include the ammonium, potassium, sodium, calcium and magnesium salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, trimethamine, dicyclohexylamine, lysine, arginine, histidine, caffeine, procaines, hydrabamine, choline, betaine, ethylenediamine, glucosamine, methylglucamine, theobromine, purines, piperazine, piperidine, N-ethylpiperidine, and the like.

Briefly, the compounds of the invention derive from a new¹ class of inhibitors of the metalloenzyme carbonic anhydrase (CA, EC 4.2.1.1)², the coumarins.³

In this patent we describe classes of coumarins that are newly found to be highly efficient, potent, isoform-selective CA IX/XII inhibitors, which also demonstrate efficacy in vivo in reducing the growth of primary tumors and metastases.

The compounds of the invention are useful for the preparation of medicaments as well as in a method for the treatment of a hypoxic tumor that has CAIX or CAXII highly overexpressed. The medicament has inhibiting action toward CAIX, and particularly it is effective for reversing acidification of a hypoxic tumor and its surrounding environment.

The compounds of the invention may be compounded with known pharmaceutical excipients such as salts, water, lipids, and/or simple sugars to arrive at a formulation suitable for injection, topical application, or ingestion.

Pharmaceutically acceptable excipients make a chemical compound stable, tolerable and acceptable for human use. Half-life in circulation can be increased, or better biodistribution achieved, by use of pharmaceutical excipients. Formulations of the compounds including pharmaceutical excipients are devised, refined, and tested during the preclinical stage of drug development to ensure that the drug is compatible with any solubilizing, stabilizing, lyophilizing, or hydrating agents.

The design of any formulation involves the characterization of a drug's physical, chemical, and mechanical properties in order to choose what other ingredients should be used in the preparation.

Particle size, polymorphism, pH, and solubility, as all of these can influence bioavailability and hence the activity of a drug. The drug must be combined with inactive additives by a method which ensures that the quantity of drug present is consistent in each dosage unit e.g. each tablet.

By the time phase III clinical trials are reached, the formulation of the drug should have been developed to be close to the preparation that will ultimately be used in the market. Stability studies are carried out to test whether temperature, humidity, oxidation, or photolysis (ultraviolet light or visible light) have any effect, and the preparation is analysed to see if any degradation products have been formed.

In one embodiment, the compounds of the invention are formulated in polyethyleneglycol with ethanol and saline. In one particular embodiment, the formulation consists of 37.5% PEG400, 12.5% ethanol and 50% saline.

As used in this document, tumor may be taken to mean any primary or metastatic cancer, hypoxic tumor tissue, or malignant growth. Any tumor susceptible to hypoxia and/or metastases, particularly breast, lung, renal cancers, cervical, pancreatic, colorectal, glioblastoma, prostate and ovarian cancer may be treated according to embodiments of the invention.

Tumors susceptible to treatment will have elevated levels of CAIX or CAXII with respect to normal tissue. As demonstrated in the data, CAIX and CAXII are associated with hypoxia and metastases. Thus a hypoxic and metastatic tumor would not need to be tested to prove elevated levels of CAIX and CAXII to indicate treatment using the compounds of the invention because of the data already supporting the supposition.

Tumor growth and/or spread may be said to be suppressed by compounds of the invention, or by their use. Suppression in this application may mean induction of regression, inhibition of growth, and inhibition of spread, especially as these terms relate to tumors and cancers suffered by mammals, particularly humans.

Typical chemotherapeutic agents including, but not limited to docetaxel, vinca alkaloids, mitoxanthrone, cisplatin, paclitaxel, 5-FU, Herceptin, Avastin, Gleevec may be used concommitally or in combination with the compounds of the invention.

Compounds of the invention may be used preoperatively, perioperatively, or post-operatively. Dosage is typically determined by dosing schemes which use patient size and weight to calculate the patient's body surface area, which correlates with blood volume, to determine initial dosing. Starting dosages are generally worked out during clinical testing of therapeutic compounds.

The background and current approaches for the clinical approach to tumor treatment may be found in Takimoto C H, Calvo E. “Principles of Oncologic Pharmacotherapy” in Pazdur R, Wagman L D, Camphausen K A, Hoskins W J (Eds) Cancer Management: A Multidisciplinary Approach. 11 ed. 2008, which is available at www.cancernetwork.com/cancer-management-11/chapter03/article/10165/1402628.

The following examples are used to illustrate aspects of the invention, but the invention is not limited to these illustrations.

### EXAMPLES

### Example 1

### Synthetic Routes for 4-Substituted Glycosylated Coumarin

### Synthesis of 4-methylumbellifer-7-yl-α-D-mannopyranoside (6) (MST-204)

Synthesis was done following and adapting procedures described by Penverne, C. and Ferrières, V. in Synthesis of 4-Methylumbellifer-7-yl-alpha-D
Mannopyranoside: An Introduction to Modern Glycosylation Reactions J. Chem. Educ., 2002, 79 (11), p 1353.

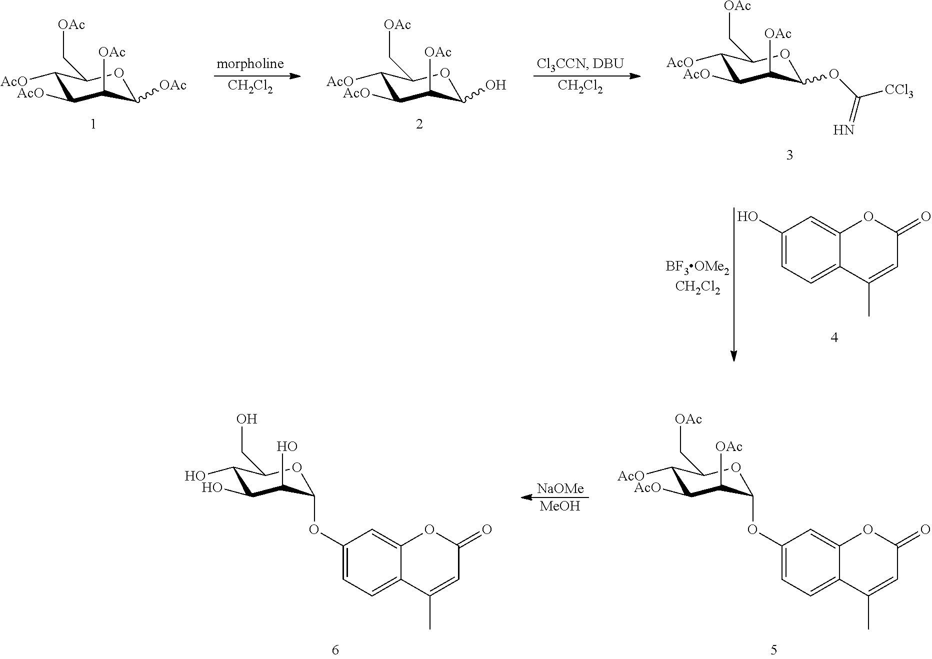

Although this example is given in the case of the mannose, similar procedures may be used for the synthesis of other sugar derivatives such as those shown below (glucose, galactose, rhamnose, xylose, sucrose, and ribose). Numbering (1, 2, 3, etc.) in the examples below is based on the numbering in the general synthetic scheme preceding this paragraph.

As illustrated schematically above, D-mannose pentaacetate (1) (10.25×10⁻³ mol) was dissolved in dry CH₂Cl₂ (40 ml). Morpholine (41×10⁻³ mol) was then added, and the mixture was stirred under N₂ atmosphere at room temperature over night. The mixture was then washed twice with 40 ml of HCl 1N and 3×20 ml of water, dried (MgSO₄) and concentrated under vacuum to give the 2,3,4,6-tetra-O-acetyl-D-mannopyranose (2).

Compound 2,3,4,6-tetra-O-acetyl-D-mannopyranose (2) (4.31×10⁻³ mol) was dissolved in dry CH₂Cl₂ (38 ml). Trichloacetonitrile (43.1×10⁻³ mol) was added, and the mixture was stirred under N₂ atmosphere at 0° C. for 1 h. Then diazabicyclo [5.4.0]undec-7-ene (DBU) (0.86×10⁻³ mol) was added and the mixture was stirred under N₂ atmosphere at 0° C. for 30 min and concentrated under vacuum. The crude 2,3,4,6-tetra-O-acetyl-D-mannopyranosyl trichloroacetimidate (3) was used without further purification in the next step.

The crude 2,3,4,6-tetra-O-acetyl-D-mannopyranosyl trichloroacetimidate (3) (4.31×10⁻³ mol) was dissolved in dry CH₂Cl₂ (38 ml). 7-hydroxy-4-methyl coumarin (4) (4.31×10⁻³ mol) and boron trifluoride metherate (BF₃.Me₂O) (0.86×10⁻³ mol) were then added and the mixture was stirred under N₂ atmosphere at room temperature over night. 20 ml of CH₂Cl₂ were further added and the solution was washed with water, dried over anhydrous MgSO₄ and concentrated under vacuum. The crude product (5) was then purified by crystallization from MeOH or by silica gel column chromatography (EP/AcOEt v/v 5/5).

The 2,3,4,6-tetra-O-acetyl-α-D-mannopyranosyl coumarin (5) (0.59×10⁻³ mol) was added to a solution of MeONa (0.88×10⁻³ mol) in dry MeOH (5 ml). The mixture was stirred at room temperature for 30 min. The product (6) was then purified by crystallization or by silica gel column chromatography (EP/AcOEt v/v 5/5) to provide:

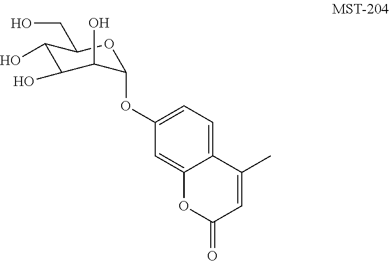 [SMILES: Cc1cc(=O)oc2cc(OC3OC(CO)C(O)C(O)C3O)ccc12]

### Characterization

Overall yield: 51%; Rf: 0.24 (CH₂Cl₂/MeOH 9/1). mp: 132-134° C. ¹H-NMR (400.13 MHz, DMSO-d6) δ ppm: 2.4 (d, 3H, J=0.8 Hz), 3.33 (m, 1H), 3.47 (m, 1H), 3.51 (t, 1H, J=9.4 Hz), 3.57 (m, 1H), 3.69 (dd, 1H, J=9.2 Hz), 3.86 (d, 1H, J=1.2 Hz), 5.53 (d, 1H, J=1.6 Hz), 6.24 (d, 1H, J=1.2 Hz), 7.09 (d, 1H, J=2.4 Hz), 7.11 (dd, 1H, J=8.8 Hz, J=2.4 Hz); 7.70 (d, 1H, J=8.8 Hz). ¹³C-NMR (100 MHz, DMSO-d6) δ ppm 18.82, 61, 66.95, 70.43, 71, 76.06, 99.48, 104.31, 112.38, 114.38, 114.79, 127.14, 160.80, 159.83, 155.02, 154.05. MS (ESI⁺) m/z: 339.24 [M+H]⁺; 361.29 [M+Na]⁺; 699.37 [2M+Na]⁺. Anal. Calcd. for C₁₆H₁₈O₈: C, 56.80; H, 5.36. Found: C, 56.84; H, 5.33.

### 4-methylumbellifer-7-yl- -L-rhamnopyranoside (MST-205)

Synthesized using a similar route to that used for MST-204.

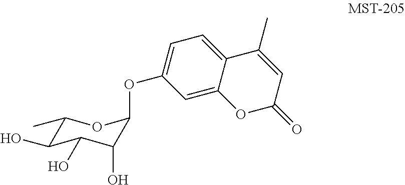 [SMILES: Cc1cc(=O)oc2cc(OC3OC(C)C(O)C(O)C3O)ccc12]

### Characterization:

Overall Yield: 58%; Rf: 0.4 (CH₂Cl₂/MeOH 9/1). mp: 207-209° C. ¹H-NMR (400.13 MHz, CDCl₃): δ ppm 1.14 (d, 3H, J=6.4 Hz), 2.35 (d, 1H, J=1.2 Hz), 3.86 (q, 1H, J=5.3 Hz), 5.10 (t, 1H, J=10 Hz), 5.42 (d, 1H, J=3.6 Hz), 5.44 (t, 1H, J=2.3 Hz, H₂), 5.45 (t, 1H, J=2.2 Hz), 6.13 (d, 1H, J=0.8 Hz), 7.02 (d, 1H, J=2.4 Hz), 7.06 (dd, 1H, J=8.8 Hz, J=2.4 Hz), 7.47 (d, 1H, J=8.8 Hz). ¹³C-NMR (100 MHz, CDCl₃): δ ppm 21.05, 21.11, 69, 69.27, 69.51, 70.1, 95, 104.26, 113.23, 113.61, 125, 152.52, 155.10, 158.61, 170.15, 170.31. MS (ESI⁺) m/z: 345.31 [M+Na]⁺; 667.39 [2M+Na]⁺. Anal. Calcd. for C₁₆H₁₈O₇: C, 59.62; H, 5.63. Found: C, 59.58; H, 5.65.

### 4-methylumbellifer-7-yl- -D-ribopyranoside (MST-207)

Synthesized using a similar route to that used for MST-204.

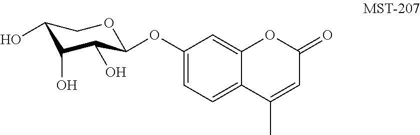

### Charcterization:

Overall Yield: 60%; Rf: 0.45 (AcOEt/MeOH 8/2). ¹H-NMR (400.13 MHz, DMSO-d6): δ ppm 2.38 (d, 3H, J=1.2 Hz); 3.91 (m, 1H), 4.03 (m, 1H), 4.70 (t, 1H, J=5.4 Hz); 5.07 (d, 1H, J=6 Hz), 5.61 (d, 1H, J=2 Hz), 6.23 (s, 1H), 6.77 (d, 1H, J=2 Hz), 6.96 (dd, 1H, J=8.4 Hz, J=2 Hz), 7.68 (d, 1H, J=8.4 Hz). ¹³C-NMR (100 MHz, DMSO-d6): δ ppm 18.09, 62.518, 70.40, 74.46, 84.81, 103.27, 105.05, 111.55, 113.36, 113.84, 126.46, 153.32, 155.3, 159.32, 160.05. MS (ESI⁺) m/z: 331.26 [M+Na]⁺, 639.25 [2M+Na]⁺. Anal. Calcd. for C₁₅H₁₆O₇: C, 58.44; H, 5.23. Found: C, 58.40; H, 5.25.

### 4-methylumbellifer-7-yl- -D-glucopyranoside (MST-202)

Synthesized using a similar route to that used for MST-204.

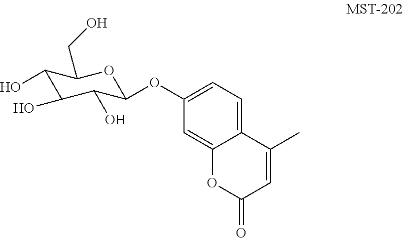 [SMILES: Cc1cc(=O)oc2cc(OC3OC(CO)C(O)C(O)C3O)ccc12]

Overall Yield: 55%; Rf: 0.39 (CH₂Cl₂/MeOH 8/2). mp: 210-212° C. ¹H-NMR (400.13 MHz, DMSO-d6): δ ppm 2.41 (s, 3H), 3.17 (dd, 1H, J=14.2 Hz, J=8.8 Hz); 3.29 (dd, 2H, J=11.9 Hz, J=7.4 Hz), 3.40-3.53 (m, 2H), 5.08 (d, 1H, J=5.3 Hz), 6.25 (s, 1H), 7.03 (d, 1H, J=2.4 Hz), 7.05 (dd, 1H, J=9.2 Hz, J=2.4 Hz), 7.71 (d, 1H, J=9.2 Hz). ¹³C-NMR (100 MHz, DMSO-d6): δ ppm 18.35, 60.86, 69.85, 73.35, 76.70, 77.36, 100.21, 103.42, 111.92, 113.60, 114.29, 126.63, 153.56, 154.61, 160.33, 160.37. MS (ESI⁺) m/z: 361.38 [M+Na]⁺. Anal. Calcd. for C₁₆H₁₈O₈: C, 56.80; H, 5.36. Found: C, 56.85; H, 5.41.

### 4-methylumbellifer-7-yl-1-D-galactopyranoside (MST-203)

Synthesized using a similar route to that used for MST-204.

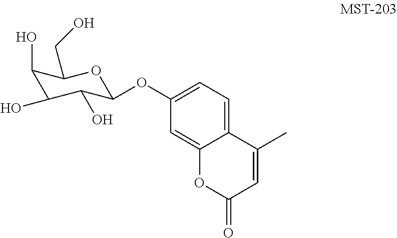 [SMILES: Cc1cc(=O)oc2cc(OC3OC(CO)C(O)C(O)C3O)ccc12]

### Characterization:

Overall Yield: 64%; Rf: 0.35 (CH₂Cl₂/MeOH 8/2). mp: 248° C. ¹H-NMR (400.13 MHz, DMSO-d6): δ ppm 2.41 (s, 3H), 3.44 (ddd, 1H, J=9.2 Hz, J=5.5 Hz, J=3.3 Hz), 3.48-3.65 (m, 3H), 3.68 (t, 1H, J=6.3 Hz), 3.72 (t, 1H, J=3.8 Hz), 4.99 (d, 1H, J=7.7 Hz), 6.25 (s, 1H), 7.02 (d, 1H, J=2.4 Hz), 7.05 (dd, 1H, J=9.1 Hz, J=2.4 Hz); 7.70 (d, 1H, J=9.1 Hz). ¹³C-NMR (100 MHz, DMSO-d6): δ ppm 18.15, 60.39, 68.13, 69.87, 73.22, 75.71, 100.60, 103.15, 112.24, 112.85, 114.79, 126.17, 153.89, 154.75, 160.19, 160.19. MS (ESI⁺) m/z: 361.35 [M+Na]⁺. Anal. Calcd. for C₁₆H₁₈O₈: C, 56.80; H, 5.36. Found: C, 56.75; H, 5.31.

### 4-methylumbellifer-7-yl- -D-xylopyranoside (MST-201)

Synthesized using a similar route to that used for MST-204.

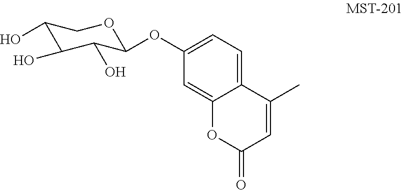 [SMILES: Cc1cc(=O)oc2cc(OC3OCC(O)C(O)C3O)ccc12]

Overall Yield: 45%; Rf: 0.58 (CH₂Cl₂/MeOH 8/2). mp: 223° C. ¹H-NMR (400.13 MHz, DMSO-d6): δ ppm 2.40 (s, 3H); 3.27 (d, 2H, J=2.3 Hz), 3.40 (m, 2H), 3.76 (m, 1H), 5.12 (d, 1H, J=3.9 Hz), 6.25 (s, 1H), 7.01 (d, J=2.4 Hz, 1H), 7.03 (dd, 1H, J=9.2 Hz, J=2.4 Hz), 7.70 (d, J=9.2 Hz, 1H). ¹³C-NMR (100 MHz, DMSO-d6): δ ppm 18.13, 62.73, 69.27, 72.95, 76.32, 100.32, 102.74, 112.74, 113.36, 114.13, 126.47, 153.32, 155.3, 159.32, 160.05. MS (ESI⁺) m/z: 331.32 [M+Na]⁺. Anal. Calcd. for C₁₅H₁₆O₇: C, 58.44; H, 5.23. Found: C, 58.49; H, 5.20.

### 4-methylumbellifer-7-yl- -D-melibiopyranoside (MST-206)

Synthesized using a similar route to that used for MST-204.

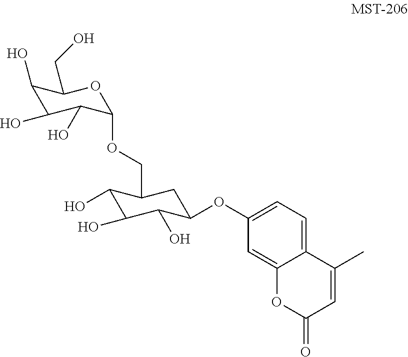 [SMILES: Cc1cc(=O)oc2cc(OC3CC(COC4OC(CO)C(O)C(O)C4O)C(O)C(O)C3O)ccc12]

### Characterization:

Overall Yield: 47%; Rf: 0.1 (AcOEt/MeOH 8/2). mp: 103-105° C. ¹H-NMR (400.13 MHz, DMSO-d6): δ ppm 2.41 (s, 3H), 3.18 (dd, 1H, J=25.6 Hz, J=13.2 Hz), 3.32 (m, 3H), 3.40 (dd, 2H, J=10.7 Hz, J=6.3 Hz), 3.55 (m, 6H), 4.65 (d, 1H, J=3.4 Hz), 5.00 (d, 1H, J=7.3 Hz), 6.26 (s, 1H), 7.04 (d, 1H, J=2.4 Hz), 7.10 (dd, 1H, J=8.8 Hz, J=2.4 Hz); 7.71 (d, 1H, J=8.8 Hz). ¹³C-NMR (100 MHz, DMSO-d6): δ ppm 20.66, 59.99, 60.08, 68.25, 68.35, 69.88, 70.09, 71.14, 74.32, 75.05, 77.26, 98.89, 100.02, 104.67, 111.38, 112.53, 114.23, 126.55, 154.17, 160.28, 166.57, 173.79. MS (ESI⁺) m/z: 523.16 [M+Na]⁺. Anal. Calcd. for C₂₂H₂₈O₁₃: C, 52.80; H, 5.64. Found: C, 52.75; H, 5.61.

### Esculin (MST-208)

### Available from Chemical Retailers)

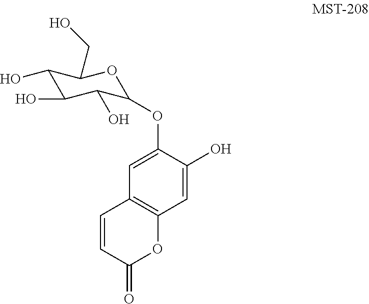 [SMILES: O=c1ccc2cc(OC3OC(CO)C(O)C(O)C3O)c(O)cc2o1]

### 4-methylumbelliferyl-beta-D-glucuronide (MST-209)

### Available from Chemical retailers

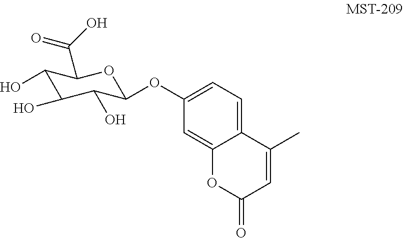 [SMILES: Cc1cc(=O)oc2cc(OC3OC(C(=O)O)C(O)C(O)C3O)ccc12]

### Example 3

### Synthetic Routes for Other Glycosylated Thiocoumarins

The Huisgen reaction is a very versatile chemical transformation consistent in the coupling of an alkyne or alkene, as diapolarophile, and a 1,3-dipolar compound such as an azide, nitriloxide and diazoalkane.

The coupling of an acetylenic coumarin/thiocoumarin scaffold with phenylazide (Scheme 1 below) and an azido coumarin with acetilenic compounds (Scheme 2 below) via a copper catalyzed reaction is shown.

The syntheses were carried out adapting the procedures reported in Brant C. Boren, Sridhar Narayan, Lars K. Rasmussen, et al., J. Am. Chem. Soc., 2008, 130, 8923-8930; Li Zhang, Xinguo Chen, Peng Xue, et al., J. Am. Chem. Soc., 2005, 127, 15998-15999; Herna'n A. Orgueira,* Demosthenes Fokas, Yuko (some, et al., Tet. Lett., 2005, 46, 2911-2914; Giancarlo Cravotto, Gianni Balliano, Silvia Tagliapietra, et al., Eur. J. of Med. Chem., 2004, 39, 917-924; Jacob Kofoed, Tamis Darbre and Jean-Louis Reymond, Org. Biomol. Chem., 2006, 4, 3268-3281; and Andrew Fryer in PCT publication WO 2008/147764 A1.

### 7-hydroxy coumarin and 7-amino coumarin scaffolds

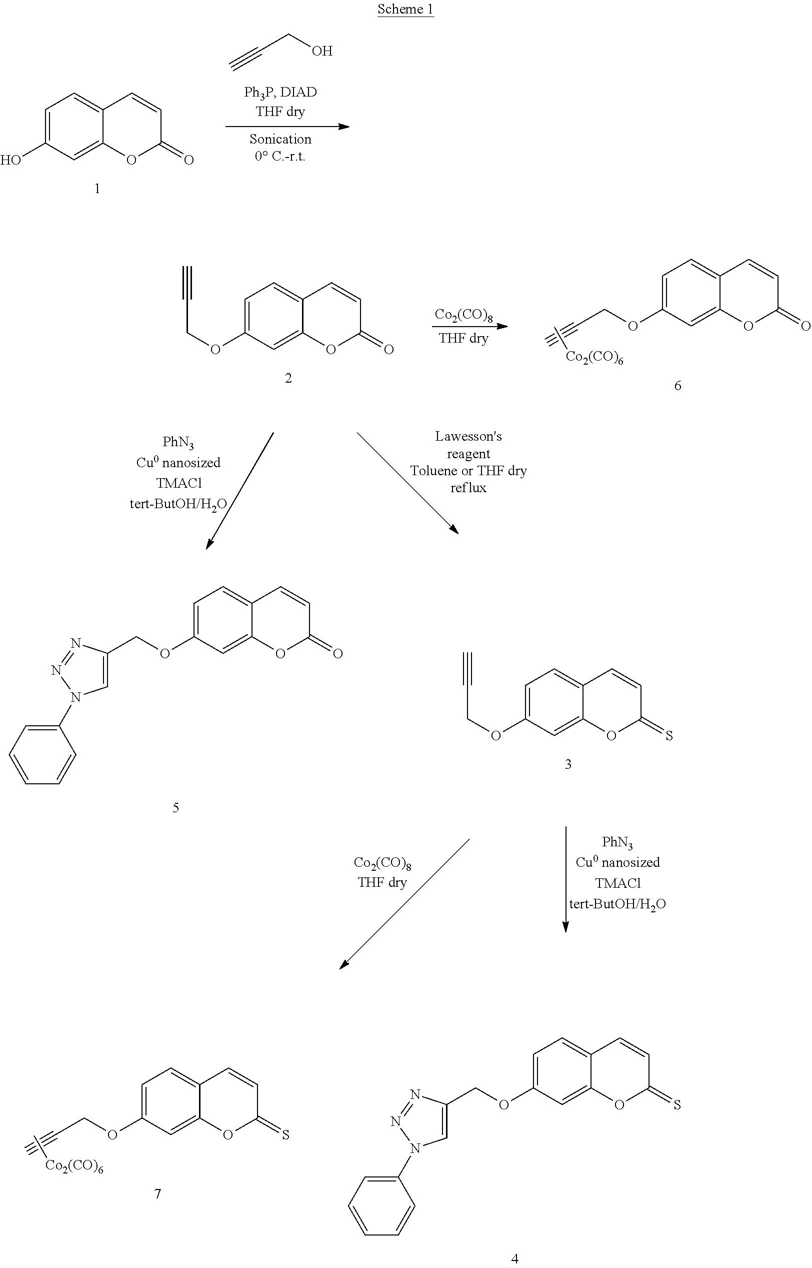

### Synthesis of 7-(prop-2-ynyloxy)-2H-chromen-2-one 2)

7-Hydroxy coumarin 1 (1.0 g, 1.0 eq), propargyl alcohol (1.0 eq) and triphenylphoshine (1.0 eq) were dissolved in dry THF (90 ml). Then the temperature was lowered to 0° C. and diisopropylazadicarboxylate (1.1 eq) was added drop-wise under sonication. The orange solution was sonicated at r.t. under a nitrogen atmosphere until starting material was consumed (TLC monitoring). Solvents were removed under vacuo to give a white solid that was recrystallized from MeOH to give 2 as white solid.

### Characterization:

7-(Prop-2-ynyloxy)-2H-chromen-2-one 2: m.p. 118° C. (Lit 120° C.); Page: 31 Rodighiero, P.; Manzini, P.; Pastorini, G.; Bordin, F.; Guiotto, A., Journal of Heterocyclic Chemistry, 24, 2, 485-8. silica gel TLC R_{f} 0.53 (Ethyl Acetate/n-hexane 50% v/v); vₘₐₓ (KBr) cm⁻¹, 3310 (C≡C—H), C2160 (C≡CH), 1765 (C═O), 1604 (Aromatic); δ_{H} (400 MHz, DMSO-d₆) 3.69 (1H, t, J 2.4, 3′-H), 4.97 (2H, d, J 2.4, 1′-H₂), 6.36 (1H, d, J 9.6, 3-H), 7.03 (1H, dd, J 8.5, 2.3, 6-H), 7.09 (1H, d, J 2.3, 8-H), 7.69 (1H, d, J 8.5, 5-H), 8.03 (1H, d, J 9.6, 4-H); δ_{C} (100 MHz, DMSO-d₆) 161.1 (C-2), 161.0 (C-7), 156.0 (C-8a), 145.1 (C-4), 130.4 (C-5), 113.9 (C-3), 113.8 (C-4-a), 113.7 (C-6), 102.7 (C-8), 79.8 (C-2′), 79.4 (C-3′) and 57.0 (C-1′).

### Synthesis of 7-(prop-2-ynyloxy)-2H-chromene-2-thione 3

7-(Prop-2-ynyloxy)-2H-chromen-2-one 2 (0.2 g, 1.0 eq) and Lawesson's Reagent (1.5 eq) were dissolved in dry toluene (10 ml) and the yellow solution was refluxed until starting material was consumed (TLC monitoring). Then the solvent was removed under vacuo and the orange residue was partitioned between H₂O and ethyl acetate. The organic phase was washed with H₂O (2×20 ml), brine (3×20 ml), dried over Na₂SO₄, filtered off and concentrated under vacuo to give a red sticky oil that was purified by silica gel column chromatography eluting with 10% ethyl acetate in n-hexane to give 3 as a yellow solid.

### Characterization:

7-(Prop-2-ynyloxy)-2H-chromene-2-thione 3: m.p. 97-101° C.; silica gel TLC R_{f} 0.27 (Ethyl Acetate/n-hexane 10% v/v); vₘₐₓ (KBr) cm⁻¹, 3300 (C≡C—H), 2165 (C≡CH), 1601 (Aromatic); δ_{H} (400 MHz, DMSO-d₆) 3.72 (1H, t, J 2.4, 3′-H), 5.02 (2H, d, J 2.4, 1′-H₂), 7.13 (1H, dd, J 9.2, 2.4, 6-H), 7.18 (1H, d, J 9.2, 3-H), 7.31 (1H, d, J 2.4, 8-H), 7.80 (1H, d, J 9.2, 5-H), 7.90 (1H, d, J 9.2, 4-H); δ_{C} (100 MHz, DMSO-d₆) 198.1 (C-2), 161.8 (C-7), 158.6 (C-8a), 137.4 (C-4), 130.6 (C-5), 127.4 (C-3), 115.7 (C-4-a), 115.6 (C-6), 102.3 (C-8), 80.0 (C-2′), 79.2 (C-3′) and 57.3 (C-1′). Anal. Calc %. C, 66.65; H, 3.73; S, 14.83; Anal. Found. C, 65.36; H, 3.71; S, 9.37.

### Synthesis of 7-[(1-phenyl-1H-1,2,3-triazol-4-yl)methoxy]-2H-chromene-2-thione 4

7-(Prop-2-ynyloxy)-2H-chromene-2-thione 3 (0.1 g, 1.0 eq) and phenylazide (1.1 eq) were dissolved in tert-ButOH/H₂O (1/1, 2.0 ml). Then tetramethylamonium chloride (1.0 eq) and copper nanosize (10% mol) were added. The mixture was vigorously stirred at r.t. until starting material was consumed (TLC monitoring). Solvents were removed under vacuo (temperature has not to exceed 40° C.) and the brown residue was purified by silica gel column chromatography eluting with 50% ethyl acetate in n-hexane to give 4 as a yellow solid.

Characterization: 7-[(1-Phenyl-1H-1,2,3-triazol-4-yl)methoxy]-2H-chromene-2-thione 4: silica gel TLC R_{f} 0.50 (Ethyl Acetate/n-hexane 10% v/v); vₘₐₓ (KBr) cm⁻¹, 1604 (Aromatic); δ_{H} (400 MHz, DMSO-d₆) 5.50 (2H, s, 1′-H₂), 7.12 (1H, dd, J 9.6, 2.4, 6-H), 7.26 (1H, d, J 9.6, 3-H), 7.35 (1H, d, J 2.4, 8-H), 7.58 (1H, tt, J 7.6, 1.2, Ar—H), 7.70 (2H, d, J 7.6, 2×Ar—H), 7.72 (1H, d, J 9.6, 5-H), 7.95 (2H, d, J 7.6, 2×Ar—H), 8.02 (1H, d, J 9.6, 4-H), 9.01 (1H, s, 3′-H); δ_{C} (100 MHz, DMSO-d₆) 198.0 (C-2), 162.0 (C-7), 157.0 (C-8a), 146.3 (C-2′), 144.0 (C-4), 136.0, 132.0, 131.0, 1230, 124.6, 121.0, 115.0, 114.0, 113.7, 103.0 (0-8) and 63.0 (C-1′).

### Synthesis of 7-[(1-phenyl-1H-1,2,3-triazol-4-yl)methoxy]-2H-chromen-2-one)

7-(Prop-2-ynyloxy)-2H-chromen-2-one 2 (0.08 g, 1.0 eq) and phenylazide (1.1 eq) were dissolved in tert-ButOH/H₂O (1/1, 2.0 ml) and then tetramethylamonium chloride (1.0 eq) and copper nanosize (5% mol) were added. The mixture was vigorously stirred at r.t. until starting material was consumed (TLC monitoring). Solvents were removed under vacuo (temperature has not to exceed 40° C.) and the brown residue was purified by silica gel column chromatography eluting with 25% ethyl acetate in n-hexane to give 5 as a white solid.

Characterization: 7-[(1-Phenyl-1H-1,2,3-triazol-4-yl)methoxy]-2H-chromen-2-one 5: m.p. 170-174° C. silica gel TLC R_{f} 0.11 (Ethyl Acetate/n-hexane 25% v/v); v_{max (KBr) cm}⁻¹ 1750 (C═O), 1602 (Aromatic); δ_{H} (400 MHz, DMSO-d₆) 5.40 (2H, s, 1′-H₂), 6.35 (1H, d, J 9.6, 3-H), 7.10 (1H, dd, J 9.6, 2.4, 6-H), 7.24 (1H, d, J 2.4, 8-H), 7.55 (1H, tt, J 7.6, 1.2, Ar—H), 7.65 (2H, d, J 7.6, 2×Ar—H), 7.7 (1H, d, J 9.6, 5-H), 7.95 (2H, d, J 7.6, 2×Ar—H), 8.04 (1H, d, J 9.6, 4-H), 9.04 (1H, s, 3′-H); δ_{C} (100 MHz, DMSO-d₆) 162.0 (C-2), 161.2 (C-7), 156.2 (C-8a), 145.2 (C-2′), 144.1 (0-4), 138.0, 130.9, 130.5, 129.8, 124.1, 121.2, 113.8, 113.7, 113.6, 102.6 (0-8) and 63.0 (C-1′).

### Synthesis of 7-(prop-2-ynyloxy)-2H-chromen-2-one hexacarbonyldicobalt 6 (MST-224)

7-(Prop-2-ynyloxy)-2H-chromen-2-one 2 (0.1 g, 1.0 eq) was dissolved in THF (10 ml) and then cobalt carbonyl (1.05 eq) was added. The black solution was stirred at r.t. for 40 min. Then SiO₂ (0.3 g) was added and solvent removed under vacuo to give a black solid that was purified by silica gel column chromatography eluting with 20% ethyl acetate in n-hexane to give 6 as a red solid.

Characterization: 7-(Prop-2-ynyloxy)-2H-chromen-2-one hexacarbonyldicobalt 6: silica gel TLC R_{f} 0.22 (Ethyl Acetate/n-hexane 20% v/v); vₘₐₓ (KBr) cm⁻¹ 1752 (C═O), 1600 (Aromatic); δ_{H} (400 MHz, DMSO-d₆) 5.50 (2H, s, 1′-H₂), 6.35 (1H, d, J 9.4, 3-H), 6.89 (1H, s, 3′-H), 7.00 (1H, dd, J 8.8, 2.4, 6-H), 7.11 (1H, d, J 2.4, 8-H), 7.70 (1H, d, J 8.8, 5-H), 8.04 (1H, d, J 9.4, 4-H); δ_{C} (100 MHz, DMSO-d₆) 200.9 (C═O), 161.7 (C-2), 161.0 (C-7), 156.2 (C-8a), 145.1 (C-4), 130.5 (C-5), 113.7, 113.6, 113.4, 102.4 (C-8), 90.8 (C-3′), 73.9 and 69.4.

### Synthesis of 7-(prop-2-ynyloxy)-2H-chromene-2-thione hexacarbonyldicobalt 7 (MST-216)

7-(Prop-2-ynyloxy)-2H-chromene-2-thione 3 (0.02 g, 1.0 eq) was treated with cobalt carbonyl (1.05 eq) as for the procedure for 6. The solvent removed under vacuo to gave a black solid that was purified by silica gel column chromatography eluting with 10% ethyl acetate in n-hexane affording 7 as a red solid.

Characterization: 7-(Prop-2-ynyloxy)-2H-chromene-2-thione hexacarbonyldicobalt 7: silica gel TLC R_{f} 0.13 (Ethyl Acetate/n-hexane 10% v/v); vₘₐₓ (KBr) cm⁻¹ 1750 (C═O), 1603 (Aromatic); δ_{H} (400 MHz, DMSO-d₆) 5.55 (2H, s, 1′-H₂), 6.90 (1H, s, 3′-H), 7.09 (1H, dd, J 8.8, 6-H), 7.20 (1H, d, J 9.2, 3-H), 7.36 (1H, d, J 2.4, 8-H), 7.82 (1H, d, J 8.8, 5-H), 7.90 (1H, d, J 9.2, 4-H); δ_{C} (100 MHz, DMSO-d₆) 200.7 (C═O), 198.3 (C═S), 166.5, 162.4, 158.9, 137.2, 131.0, 127.9, 115.4, 101.9, 73.9, 69.7 and 57.4; Anal. Calc %. C, 44.12; H, 2.14; S, 6.20. Anal. Found. 42.75; H, 1.22; S, 3.94.

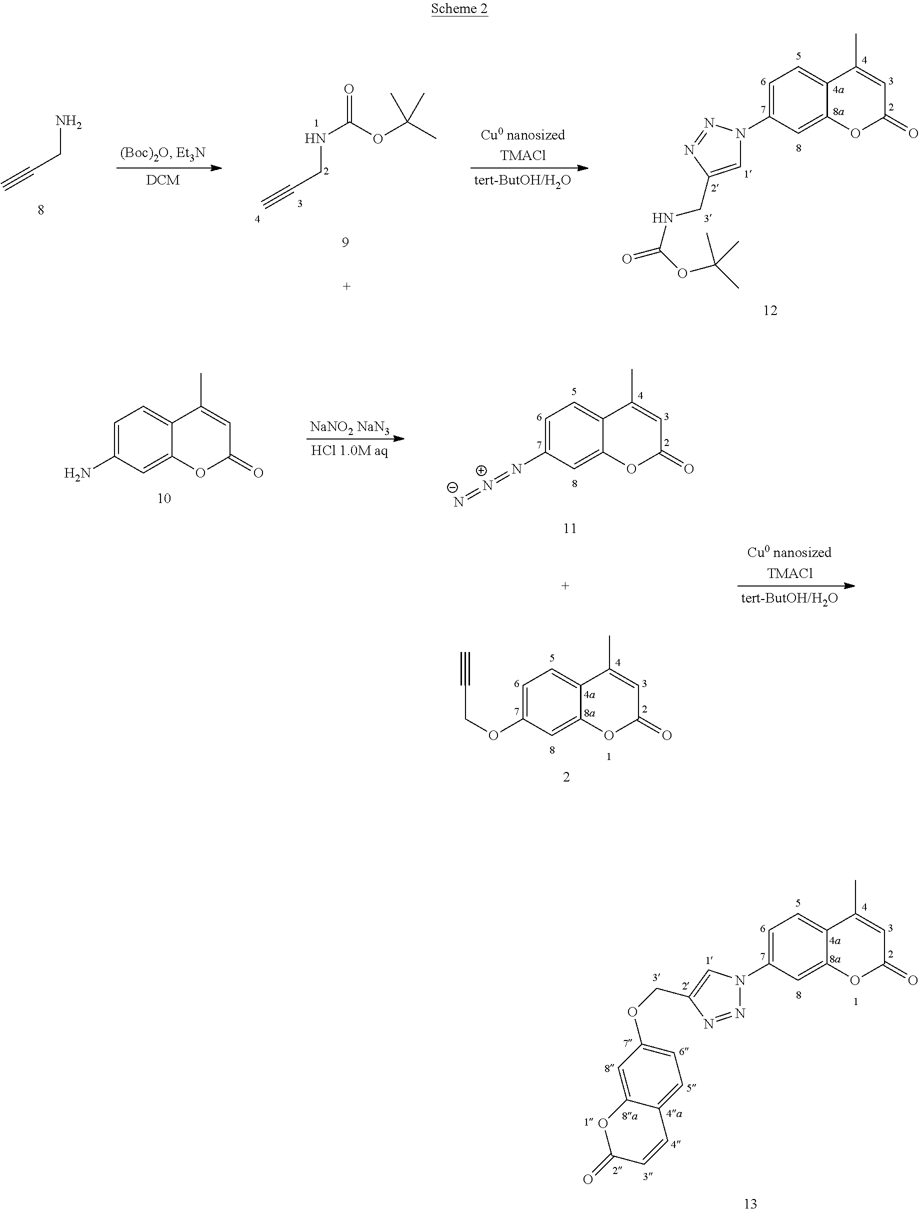

### Synthesis of tert-butyl prop-2-ynylcarbamate 9

Propargylamine 8 (1.0 g, 1.0 eq) and triethylamine (1.1 eq) were dissolved in DCM (80 ml). The solution was cooled to 0° C. and tert-butyloxycarbonylcarbonate (1.1 eq) dissolved in 20 ml of DCM was added drop-wise. The solution was stirred at r.t. for 5 h then was quenched with aqueous HCl 1.0M (100 ml) and the organic layer was washed with H2O (3×50 ml), brine (3×20 ml) and dried over Na₂SO₄, filtered off and concentrated under vacuo to give a brown oil that was purified by silica gel column chromatography eluting with 10% ethyl acetate in n-hexane to give 9 as a colorless oil.

Characterization: tert-Butyl prop-2-ynylcarbamate 9: silica gel TLC R_{f} 0.20 (Ethyl Acetate/n-hexane 10% v/v); vₘₐₓ (KBr) cm⁻¹ 3350 (C≡C—H), 2170 (C≡CH), 1760 (C═O); δ_{H} (400 MHz, DMSO-d₆) 1.42 (9H, s, 3×CH₃), 3.08 (1H, t, J 4.0, 4-H), 3.73 (2H, m, 2-H₂), 7.29 (1H, brs, 1-H); δ_{C} (100 MHz, DMSO-d₆) 156.6 (C═O), 82.6, 79.1, 73.6, 30.3 (C-2) and 29.9 (3×CH3).

### Synthesis of 7-azido-4-methyl-2H-chromen-2-one 11

7-Amino-4-methyl-2H-chromen-2-one 10 (0.1 g, 1.0 eq) was dissolved in a freshly prepared 40% solution of concentrated hydrochloric acid in deionised water (3.0 ml) and then cooled down to −5° C. Then a 2.3 M aqueous solution of NaNO₂ (2.0 eq) was added dropwise and the mixture was kept stirring at the same temperature until a persistent pale yellow solution was formed (5-10 min). Finally a 5.0 M aqueous solution of NaNO₂ (2.0 eq) was added drop-wise the mixture was stirred at r.t. for 10 min., extracted with DCM (3×25 ml) and the combined organic layers were dried over Na₂SO₄, filtered off and concentrated under vacuo (temperature has not to exceed 30° C.) to give a 11 as a yellow solid that was used without further purification.

Characterization: 7-Azido-4-methyl-2H-chromen-2-one 11: silica gel TLC R_{f} 0.27 (Ethyl Acetate/n-hexane 20% v/v); vₘₐₓ (KBr) cm⁻¹ 2150 (N₃), 1730 (C═O); δ_{H} (400 MHz, DMSO-d₆) 2.45 (9H, s, 3×CH₃), 6.37 (1H, d, J 1.2, 8-H), 7.16 (1H, dd, J 1.2, 6-H), 7.19 (1H, d, J 1.2, 3-H), 7.81 (1H, J 8.4, 5-H); δ_{C} (100 MHz, DMSO-d₆) 160.4 (C═O), 155.0, 153.8, 144.2, 127.9, 117.7, 116.5, 114.1, 107.7 and 19.0 (CH₃).

### Synthesis of tert-butyl [1-(4-methyl-2-oxo-2H-chromen-7-yl)-1H-1,2,3-triazol-4-yl]methylcarbamate 12

7-Azido-4-methyl-2H-chromen-2-one 11 (0.09 g, 1.0 eq) and tert-butyl prop-2-ynylcarbamate 9 (1.0 eq) were dissolved in tert-ButOH/H₂O (1/1, 3.0 ml) and then tetramethylamonium chloride (1.0 eq) and copper nanosize (5% mol) were added. The mixture was vigorously stirred at r.t. until starting material was consumed (TLC monitoring). Solvents were removed under vacuo (temperature has not to exceed 40° C.) and the brown residue was purified by silica gel column chromatography eluting with 50% ethyl acetate in n-hexane to give 12 as a yellow solid.

Characterization: tert-Butyl [1-(4-methyl-2-oxo-2H-chromen-7-yl)-1H-1,2,3-triazol-4-yl]methylcarbamate 12: silica gel TLC R_{f} 0.13 (Ethyl Acetate/n-hexane 50% v/v); vₘₐₓ (KBr) cm⁻¹ 1735 (C═O), 1650 (C═O); δ_{H} (400 MHz, DMSO-d₆) 1.44 (9H, s, 3×CH₃), 2.51 (3H, s, CH₃), 4.32 (2H, d, J 4,3′-H₂), 6.50 (1H, d, J 1.2, 3-H), 7.43 (1H, t, J 4, N—H), 8.02 (3H, m, 5,6,8-H), 8.81 (1H, s, 1′-H); δ_{C} (100 MHz, DMSO-d₆) 160.4, 156.5, 154.6, 153.6, 148.0, 139.5, 128.1, 122.0, 120.3, 116.3, 115.5, 108.2, 79.0, 36.5, 29.2 and 19.0

### Synthesis of 4-methyl-7-(4-((2-oxo-2H-chromen-7-yloxy)methyl)-1H-1,2,3-triazol-1-yl)-2H-chromen-2-one 13 (MST-222)

7-Azido-4-methyl-2H-chromen-2-one 11 (0.05 g, 1.0 eq) and 7-(prop-2-ynyloxy)-2H-chromen-2-one 2 (1.0 eq) were dissolved in tert-ButOH/H₂O (1/1, 1.0 ml) and then tetramethylamonium chloride (1.0 eq) and copper nanosize (5% mol) were added. The mixture was vigorously stirred at r.t. until starting material was consumed (TLC monitoring). Solvents were removed under vacuo (temperature has not to exceed 40° C.) and the brown residue was purified by silica gel column chromatography eluting with ethyl acetate in n-hexane from 20% to 50% to give 13 as a yellow solid.

Characterization: 4-Methyl-7-(4-((2-oxo-2H-chromen-7-yloxy)methyl)-1H-1,2,3-triazol-1-yl)-2H-chromen-2-one 13: silica gel TLC R_{f} 0.32 (Ethyl Acetate/n-hexane 50% v/v); vₘₐₓ (KBr) cm⁻¹ 1735 (C═O), 1730 (C═O); δ_{H} (400 MHz, DMSO-d₆) 2.11 (3H, s, CH₃), 5.44 (2H, s, 3′-H₂), 6.35 (1H, d, J 9.6, 3″-H), 6.53 (1H, d, J 1.2, 3-H), 7.11 (1H, dd, J 8.4, 2.4, 6″-H), 7.25 (1H, d, J 2.4, 8″-H), 7.71 (1H, d, J 8.4, 5″-H), 8.06 (4H, m, 5, 6, 8, 4″-H), 9.22 (1H, s, 1′-H); δ_{C} (100 MHz, DMSO-d₆) 161.9, 161.0, 160.3, 156.2, 154.3, 153.7, 145.2, 144.5, 139.3, 130.5, 128.2, 124.4, 120.6, 116.6, 115.8, 113.9, 113.7, 108.6, 108.0, 102.6, 62.5 and 32.2.

### Synthesis of 2H-thiochromen-2-one (MST-210)

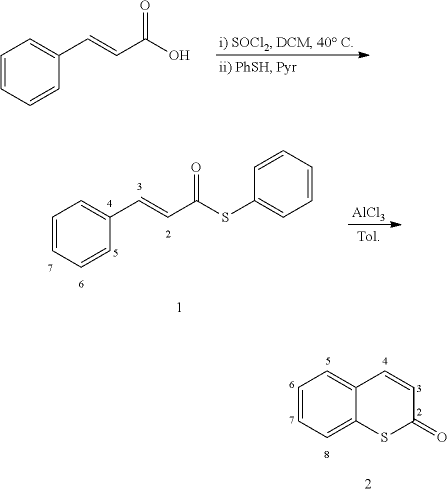

Cinnamic acid (1.0 g, 1.0 eq) was dissolved in dry DCM (20 ml) and thionyl chloride (10.0 eq) was added drop-wise at 0° C. The solution was refluxed until starting material was consumed (TLC monitoring), solvents removed under vacuo to afford a sticky oily residue that was dissolved in dry pyridine (10 ml) at 0° C. and thiophenol (0.74 g, 1.0 eq) was added drop-wise. The yellow solution was stirred at r.t. for 2 hrs, quenched with H2O (30 ml), extracted with ethyl acetate (3×15 ml) and the combined organic layers were dried over Na₂SO₄, filtered and concentrated in vacuo to give a residue that was purified by silica gel column chromatography eluting with 5% ethyl acetate/n-hexane to afford 1 a pale yellow solid.

(E)-S-phenyl 3-phenylprop-2-enethioate 1 (0.2 g, 1.0 eq) was dissolved in toluene dry (5.0 ml) and AlCl₃ (0.56 g, 5.0 eq) was added. The orange solution was stirred at 70° C. for 5 hrs (TLC monitoring), cooled down to r.t., quenched with slush and entracte with etyla acetate (3×20 ml). The combined organic layers were washed with H2O (2×20 ml), dried over Na2SO4, filtered off and concentrated in vacuo to give an orange residue that was purified by silica gel column chromatography eluting with 5% ethyl acetate/n-hexane to afford 2 as a pale yellow solid.

(E)-S-Phenyl 3-phenylprop-2-enethioate 1 62% yield; 94-96° C. (Lit 91-92° C.); silica gel TLC R_{f} 0.17 (Ethyl Acetate/n-hexane 5% v/v); vₘₐₓ (KBr) cm⁻¹, 1670 (C═O), 1515 (aromatic); δ_{H} (400 MHz, DMSO-d₆) 7.16 (1H, d, J 16.0, 2-H), 7.49 (3H, m, 2×6-H, 7-H), 7.54 (5H, s, S—Ar—H), 7.70 (1H, d, J 16.0, 3-H), 7.84 (2H, m, 2×5-H); be (100 MHz, DMSO-d₆), 188.0 (C═O), 142.5, 135.4, 134.6, 132.0, 130.5, 130.3, 130.0, 129.9, 128.2, 125.2.

2H-Thiochromen-2-one 2 55% yield; 95-98° C. (Lit 91-92° C.); silica gel TLC R_{f} 0.11 (Ethyl Acetate/n-hexane 5% v/v); vₘₐₓ (KBr) cm⁻¹, 1660 (C═O), 1515 (aromatic); δ_{H} (400 MHz, DMSO-d₆) 6.65 (1H, d, J 10.8, 3-H), 7.64 (3H, m, 5-H, 6-H, 7-H), 7.92 (1H, d, J 8.0, 8-H), 8.12 (1H, d, J 10.8, 4-H); be (100 MHz, DMSO-d₆), 185.1 (C═O), 145.8, 137.2, 133.0, 131.4, 127.8, 126.8, 126.7, 124.4; Anal. Calc. C, 66.64; H, 3.73; S, 19.77. Anal. Found. C, 62.96; H, 3.63; S, 12.08.

### General Procedure for Thionation of Lactones and Thiolactones

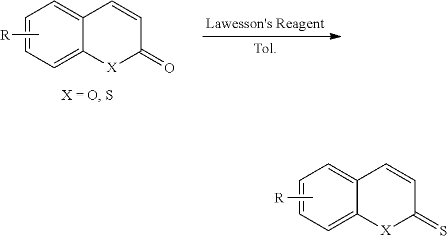

The proper lactone or thiolactone (1.0 eq) was dissolved in dry toluene and treated with Lawesson's reagent (2.0 eq). The reaction mixture was refluxed until consumption of the starting material (TLC monitoring). Then solvent was removed in vacuo and the residue obtained was purified by silica gel column chromatography eluting with ethyl acetate in n-hexane to afford the corresponding thione.

### Synthesis of 2H-thiochromene-2-thione 3 (MST-212)

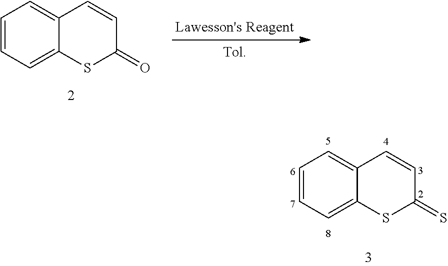 [SMILES: O=c1ccc2ccccc2s1 ; S=c1ccc2ccccc2s1]

2H-Thiochromen-2-one 2 (0.03 g, 1.0 eq) was treated according to the general procedure reported above at 70° C. for 12 h. Purification of the crude residue by silica gel column chromatography eluting with 10% ethyl acetate/n-hexane to afford the desired product 3 as a red solid.

2H-Thiochromene-2-thione 3: 33% yield; silica gel TLC R_{f} 0.20 (Ethyl Acetate/n-hexane 10% v/v); vₘₐₓ (KBr) cm⁻¹, 1770, 1520, 1230; δ_{H} (400 MHz, DMSO-d₆) 7.43 (1H, d, J 10.0, 3-H), 7.61 (1H, dt, J 8.0, 1.6, 5-H), 7.28 (2H, m, 6-H, 7-H), 7.90 (1H, d, J 10.0, 4-H), 8.00 (1H, d, J 8.0, 8-H); be (100 MHz, DMSO-d₆), 209.7 (C═S), 140.2, 136.9, 136.3, 133.0, 131.9, 129.2, 128.5, 124.6; Anal. Calc. C, 60.63; H, 3.39; S, 35.97. Anal. Found. C, 59.48; H, 3.05; S, 21.27.

### Synthesis of 2H-chromene-2-thione 4 (MST-211)

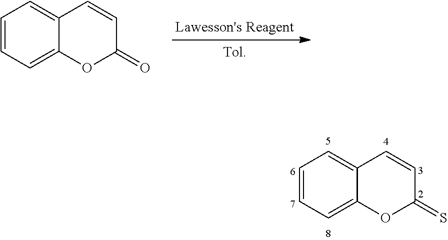 [SMILES: O=c1ccc2ccccc2o1 ; S=c1ccc2ccccc2o1]

2H-Chromen-2-one (0.5 g, 1.0 eq) was treated according to the general procedure reported above at for 12 h. Purification of the crude residue by silica gel column chromatography eluting with 20% ethyl acetate/n-hexane to afforded the desired product as a yellow solid.

2H-Chromene-2-thione 4: 60% yield; silica gel TLC R_{f} 0.27 (Ethyl Acetate/n-hexane 20% v/v); vₘₐₓ (KBr) cm⁻¹ 1765, 1518, 1220; δ_{H} (400 MHz, DMSO-d₆) 7.31 (1H, d, J 10.0, 3-H), 7.61 (1H, dt, J 7.6, 1.2, 6-H), 7.64 (1H, d, J 8.4, 5-H), 7.74 (1H, dt, J 7.6, 1.2, 7-H), 7.85 (1H, d, J 8.4, 8-H), 7.96 (1H, d, J 10.0, 4-H); be (100 MHz, DMSO-d₆), 198.5 (C═S), 157.0, 137.0, 133.6, 130.0, 129.6, 126.8, 121.2, 117.1; Anal. Calc. C, 66.64; H, 3.73; S, 19.77. Anal. Found. C, 66.15; H, 3.43; S, 12.38.

### Synthesis of 7-(allyloxy)-2H-chromene-2-thione 5 (MST-218)

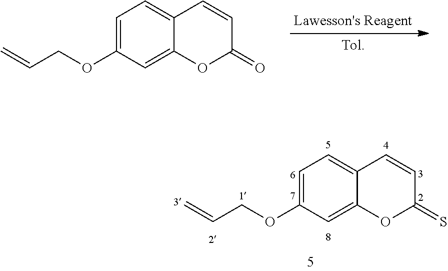

7-(Allyloxy)-2H-chromen-2-one (0.5 g, 1.0 eq) was treated according to the general procedure reported above at for 12 h. Purification of the crude residue by silica gel column chromatography eluting with 20% ethyl acetate/n-hexane to afforded the desired product as a yellow solid.

7-(Allyloxy)-2H-chromene-2-thione 5: 87% yield; silica gel TLC R_{f} 0.32 (Ethyl Acetate/n-hexane 20% v/v); vₘₐₓ (KBr) cm⁻¹ 1760, 1519, 1215; δ_{H} (400 MHz, DMSO-d₆) 4.77 (2H, dt, J 5.6, 1.6, 1′-H₂), 5.35 (1H, dq, J 12.0, 1.6, 3′-HH), 5.47 (1H, dq, J 17.2, 1.6, 3′-HH), 6.11 (1H, m, 2′-H), 7.11 (1H, dd, J 8.8, 2.4, 6-H), 7.12 (1H, d, J 9.2, 3-H), 7.27 (1H, d, J 2.4, 8-H), 7.77 (1H, d, J 8.8, 5-H), 7.88 (1H, d, J 9.2, 4-H); be (100 MHz, DMSO-d₆), 198.2 (C═S), 162.9, 158.9, 137.5, 133.7, 130.6, 127.1, 119.2, 115.8, 115.2, 102.0, 70.0; Anal. Calc. C, 66.03; H, 4.62; S, 14.69. Anal. Found. C, 66.56; H, 4.32; S, 9.68.

### Synthesis of 7-(prop-2-ynyloxy)-2H-chromene-2-thione 6 (MST-217)

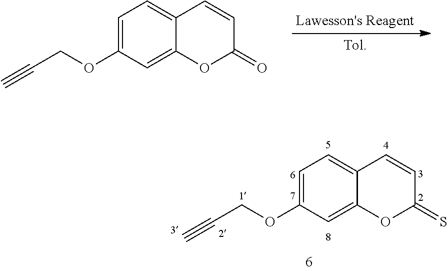

7-(Prop-2-ynyloxy)-2H-chromen-2-one (0.1 g, 1.0 eq) was treated according to the general procedure reported above at for 12 h. Purification of the crude residue by silica gel column chromatography eluting with 10% ethyl acetate/n-hexane to afforded the desired product as a yellow solid.

7-(Prop-2-ynyloxy)-2H-chromene-2-thione 6: 56% yield; silica gel TLC R_{f} 0.27 (Ethyl Acetate/n-hexane 10% v/v); Vₘₐₓ (KBr) cm⁻¹, 1762, 1523, 1210; δ_{H} (400 MHz, DMSO-d₆) 3.72 (1H, t, J 2.4, 3′-H), 5.02 (2H, d, J 2.4, 1′-H₂), 7.12 (1H, dd, J 8.8, 2.4, 6-H), 7.15 (1H, d, J 9.2, 3-H), 7.32 (1H, d, J 2.4, 8-H), 7.79 (1H, d, J 8.8, 5-H), 7.90 (1H, d, J 9.2, 4-H); be (100 MHz, DMSO-d₆), 198.1 (C═S), 161.8 (C-7), 158.6 (C-8a), 137.4 (C-4), 130.6 (C-5), 127.4 (C-3), 115.7 (C-4-a), 115.6 (C-6), 102.3 (C-8), 80.0 (C-2′), 79.2 (C-3′), 57.3 (C-1′); Anal. Calc. C, 66.65; H, 3.73; S, 14.83. Anal. Found. C, 66.36; H, 3.71; S, 9.37.

### Synthesis of tert-butyl 2-hydroxyethylcarbamate 7

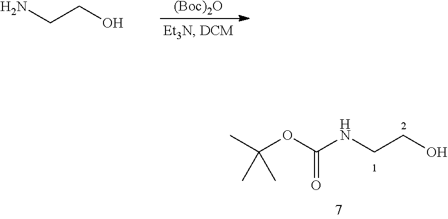

Ethanolamine (10.0 g, 1.0 eq) was dissolved in a 1.0 M NaOH aqueous solution (16.0 ml). Then a DCM solution (60 ml) of (Boc)₂O (3.93 g, 1.1 eq) was added drop wise at 0° C. under vigorous stirring. The mixture was stirred at r.t. for 1 h, quenched with 0.1M aqueous hydrochloride acid (3×20 ml), 5% NaHCO₃ aqueous solution (3×20 ml), and then washed with brine (2×20 ml), dried over Na₂SO₄, filtered off and solvent removed in vacuo to give an oily residue that was purified by silica gel column chromatography eluting with an increasing amount of MeOH in DCM from 2.5 to 5% to afford 7 a light colorless oil

tert-Butyl 2-hydroxyethylcarbamate 7: 90% yield; silica gel TLC R_{f} 0.30 (MeOH/DCM 2.5% v/v); vₘₐₓ (KBr) cm⁻¹, 3112 (O—H), 1770 (C═O); δ_{H} (400 MHz, MeOD-d₄) 7.47 (9H, s, 3×CH₃), 3.18 (2H, t, J 6.0, 2-H₂), 3.58 (2H, t, J 6.0, 1-H₂); be (100 MHz, DMSO-d₆) 26.0, 44.2. 61.9, 80.3, 157.1.

### Synthesis of tert-butyl 2-(2-oxo-2H-chromen-7-yloxy)ethylcarbamate 8 (MST-219)

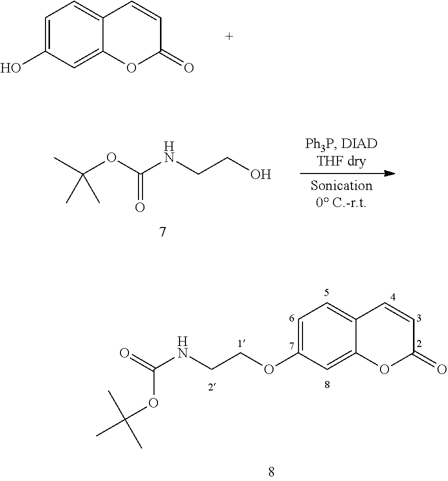 [SMILES: O=c1ccc2ccc(O)cc2o1 ; CC(C)(C)OC(=O)NCCO ; CC(C)(C)OC(=O)NCCOc1ccc2ccc(=O)oc2c1]

7-Hydroxy coumarin (0.44 g, 1.0 eq), tert-butyl 2-hydroxyethylcarbamate 7 (0.44 g, 1.0 eq) and triphenylphoshine (0.72 g, 1.0 eq) were dissolved in dry THF (60 ml). Then the temperature was lowered to 0° C. and diisopropylazadicarboxylate (0.55 g, 1.0 eq) was added drop-wise under sonication. The orange solution was sonicated at room temperature under a nitrogen atmosphere until starting material was consumed (TLC monitoring). Solvents were removed under vacuo to give a white solid that was recrystallized from H₂O/MeOH to give 8 as white solid.

tert-Butyl 2-(2-oxo-2H-chromen-7-yloxy)ethylcarbamate 8: 45% yield; silica gel TLC R_{f} 0.47 (Ethyl Acetate/n-hexane 50% v/v); vₘₐₓ (KBr) cm⁻¹, 3120, 1770 (C═O), 1520 (aromatic); δ_{H} (400 MHz, DMSO-d₆) 1.41 (9H, s, 3×CH₃), 3.32 (2H, appq, J 5.6×2′-H₂), 4.11 (2H, t, J 5.6, 1′-H₂), 6.32 (1H, d, J 9.6, 3-H), 6.97 (1H, dd, J 8.6 2.8, 6-H), 7.02 (1H, d, J 2.8, 8-H), 7.07 (1H, t, J 5.6, exchange with D₂O, NH), 7.66 (1H, d, J 8.6, 5-H), 8.03 (1H, d, J 9.2, 4-H); δ_{C} (100 MHz, DMSO-d₆), 162.6 (C═O), 161.2 (C═O), 156.6, 156.3, 145.3, 130.5, 113.7, 113.4, 103.1, 102.2, 78.8, 68.1, 29.1 (CH₃), 22.8; Anal. Calc. C, 62.94; H, 6.27; N, 4.59. Anal. Found. C, 61.90; H, 6.26; N, 4.58.

### Synthesis of 2″,4″,6″-trimethyl-1-(2-(2-oxo-2H-chromen-7-yloxy)ethyl)pyridinium perchlorate salt 9 (MST-213)

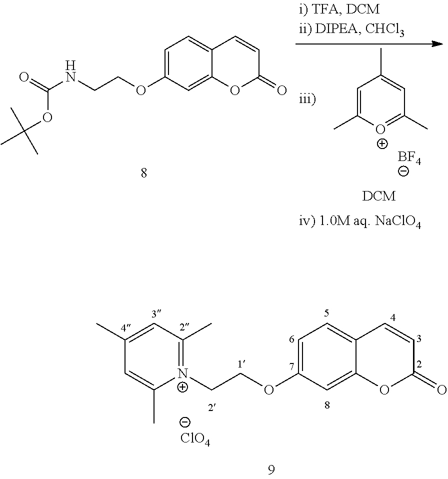

tert-Butyl 2-(2-oxo-2H-chromen-7-yloxy)ethylcarbamate 8 (0.1 g, 1.0 eq) was suspended in DCM (20 ml) and treated with TFA (5.0 eq). The yellow solution was stirred O.N. at r.t. then solvents were removed in vacuo and the white solid residue was dissolved in CHCl₃ (20 ml) and treated with DIPEA (3.0 eq). The pale yellow solution was stirred at r.t. for 1 h, diluted with H₂O (50 ml) and the organic layer was washed with brine (5×15 ml), dried over Na₂SO₄, filtered off and solvent evaporated in vacuo to give a sticky yellow oil that was dissolved in dry DCM (15 ml) and treated with 2,4,6-pyrilium tetrafluoroborate (1.5 eq) at reflux for 1 h. Then solvent was removed in vacuo and the tannic residue treated with a 1.0 M aqueous solution of NaClO₄ (3.0 eq) to give a dark precipitate that was collected by filtration and crystallized from H₂O/MeOH to afford the desired product 9 as a white solid.

2″,4″,6″-Trimethyl-1-(2-(2-oxo-2H-chromen-7-yloxy)ethyl)pyridinium perchlorate salt 9: 20% overal yield; vₘₐₓ (KBr) cm⁻¹, 3112 (O—H), 1770 (C═O), 1522 (aromatic); δ_{H} (400 MHz, DMSO-d₆) 2.53 (3H, s, 4″-CH₃), 2.93 (6H, s, 2×2″-CH₃), 4.63 (2H, t, J 4.8, 1′-H₂), 5.01 (2H, t, J 4.8, 2′-H₂), 6.35 (1H, d, J 9.2, 3-H), 6.97 (1H, dd, J 8.6 2.8, 6-H), 7.07 (1H, d, J 2.8, 8-H), 7.67 (1H, d, J 8.6, 5-H), 7.81 (2H, s, 2×3″-H), 8.02 (1H, d, J 9.2, 4-H); be (100 MHz, DMSO-d₆), 160.2, 158.2, 157.0, 152.4, 147.9, 144.2, 128.8, 128.5, 114.2. 113.9, 110.5, 109.6, 70.0, 45.2, 26.3, 22.4; Anal. Calc. C, 55.68; H, 4.92; N, 3.42. Anal. Found. C, 42.4; H, 4.93; N, 2.56.

### Synthesis of 4-methyl-N-(4-methyl-2-oxo-2H-chromen-7-yl)benzenesulfonamide 10 (MST-215)

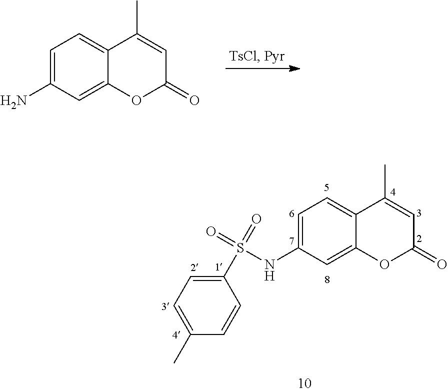 [SMILES: Cc1cc(=O)oc2cc(N)ccc12 ; Cc1ccc(S(=O)(=O)Nc2ccc3c(C)cc(=O)oc3c2)cc1]

7-Amino-4-methylcoumarin (0.1 g, 1.0 eq) was dissolved in dry pyridine (5.0 ml) and the solution cooled down to 0° C. Then tosylchloride (0.14 g, 1.3 eq) was added and the reaction mixture was stirred at r.t. until starting material was consumed (TLC monitoring). The reaction was quenched with slush, and the white precipitate formed was collected by filtration and purified by silica gel column chromatography eluting with 50% ethyl acetate/n-hexane to afford the desired product 10 as a white solid.

4-Methyl-N-(4-methyl-2-oxo-2H-chromen-7-yl)benzenesulfonamide 10: 54% yield; silica gel TLC R_{f} 0.35 (Ethyl Acetate/n-hexane 50% v/v); vₘₐₓ (KBr) cm⁻¹, 3110, 1770 (C═O), 1530 (aromatic); δ_{H} (400 MHz, DMSO-d₆) 2.37 (6H, s, 4-CH₃, 4′-CH₃), 6.27 (1H, s, 3-H), 7.06 (1H, d, J 2.0, 8-H), 7.12 (1H, dd, J 8.6, 2.0, 6-H), 7.41 (2H, d, J 8.4, 2×3′-H), 7.67 (1H, d, J 8.6, 5-H), 7.77 (2H, d, J 8.4, 2×2′-H), 10.90 (1H, brs, exchange with D₂O, NH); be (100 MHz, DMSO-d₆), 160.7 (C═O), 154.7, 154.1, 144.9, 142.4, 137.3, 131.0, 127.8, 127.6, 116.3, 115.7, 113.6, 106.1, 22.0, 18.9.

General procedure for the synthesis of acetylenehexacarbonyldicobalt complexes

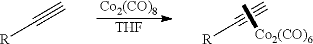

Alkine (1.0 eq) was dissolved in THF dry and then dicobaltooctacarbonyl (1.05 eq) was added. The black solution was stirred at r.t. under a nitrogen atmosphere until evolution of carbon monoxide ceased (1-2 h). Then silica gel was added and the solvent evaporated under vacuo to give a purple residue that was purified by silica gel column chromatography eluting with ethyl acetate/n-hexane to afford the corresponding acetylenehexacarbonyldicobalt complexes as reddish solids.

N.B. temperature must not exceed 30° C.

### Synthesis of 7-(prop-2-ynyloxy)-2H-chromen-2-one thione hexacarbonyldicobalt 11 (MST-224)

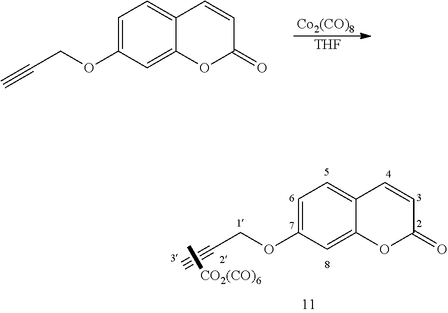

7-(Prop-2-ynyloxy)-2H-chromen-2-one (0.1 g, 1.0 eq) was dissolved in THF (10 ml) and then cobalt carbonyl (1.05 eq) was added. The black solution was treated as described above in the general procedure and the black residue obtained was purified by silica gel column chromatography eluting with 20% ethyl acetate in n-hexane to give 11 as a red solid.

7-(Prop-2-ynyloxy)-2H-chromen-2-one hexacarbonyldicobalt 11: 82% yield; silica gel TLC R_{f} 0.22 (Ethyl Acetate/n-hexane 20% v/v); vₘₐₓ (KBr) cm⁻¹ 1752 (C═O), 1600 (Aromatic); δ_{H} (400 MHz, DMSO-d₆) 5.50 (2H, s, 1′-H₂), 6.35 (1H, d, J 9.4, 3-H), 6.89 (1H, s, 3′-H), 7.00 (1H, dd, J 8.8, 2.4, 6-H), 7.11 (1H, d, J 2.4, 8-H), 7.70 (1H, d, J 8.8, 5-H), 8.04 (1H, d, J 9.4, 4-H); δ_{C} (100 MHz, DMSO-d₆) 200.9 (C═O), 161.7 (C-2), 161.0 (C-7), 156.2 (C-8a), 145.1 (C-4), 130.5 (C-5), 113.7, 113.6, 113.4, 102.4 (C-8), 90.8 (C-3′), 73.9 and 69.4.

### Synthesis of 7-(prop-2-ynyloxy)-2H-chromene-2-thione hexacarbonyldicobalt 12 (MST-216)

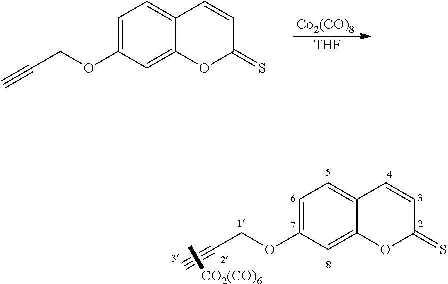

7-(Prop-2-ynyloxy)-2H-chromene-2-thione 6 (0.1 g, 1.0 eq) was dissolved in THF (10 ml) and then cobalt carbonyl (1.05 eq) was added. The black solution was treated as described above in the general procedure and the black residue obtained was purified by silica gel column chromatography eluting with 10% ethyl acetate in n-hexane to give 12 as a red solid.

7-(prop-2-ynyloxy)-2H-chromene-2-thione hexacarbonyldicobalt 12: 79% yield; silica gel TLC R_{f} 0.18 (Ethyl Acetate/n-hexane 10% v/v); vₘₐₓ (KBr) cm⁻¹ 1775 (C═O), 1530 (aromatic); δ_{H} (400 MHz, DMSO-d₆) 5.55 (2H, s, 1′-H₂), 6.90 (1H, s, 3′-H), 7.09 (1H, dd, J 8.8, 2.4, 6-H), 7.18 (1H, d, J 9.2, 3-H), 7.36 (1H, d, J 2.4, 8-H), 7.80 (1H, d, J 8.8, 5-H), 7.90 (1H, d, J 9.2, 4-H); δ_{C} (100 MHz, DMSO-d₆), 200.7 (C≡O), 198.3 (C═S), 166.5, 162.4, 158.9, 137.2, 130.0, 127.1, 115.4, 101.9, 73.9, 69.7, 57.4; Anal. Calc. C, 44.12; H, 2.14; S, 6.20. Anal. Found. C, 44.75; H, 2.08; S, 3.94.

### Synthesis of 7-(pent-4-ynyloxy)-2H-chromen-2-one hexacarbonyldicobalt 13 (MST-214)

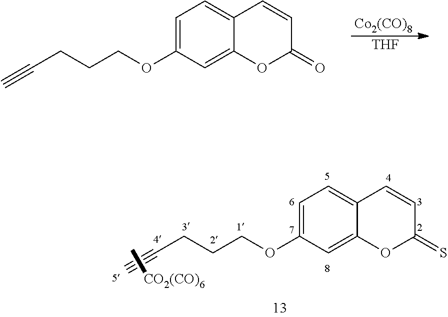

7-(Pent-4-ynyloxy)-2H-chromen-2-one (0.05 g, 1.0 eq) was dissolved in THF (10 ml) and then cobalt carbonyl (1.05 eq) was added. The black solution was treated as described above in the general procedure and the black residue obtained was purified by silica gel column chromatography eluting with 20% ethyl acetate in n-hexane to give 13 as a red solid.

7-(Pent-4-ynyloxy)-2H-chromen-2-one hexacarbonyldicobalt 13: 92% yield; silica gel TLC R_{f} 0.20 (Ethyl Acetate/n-hexane 20% v/v); vₘₐₓ (KBr) cm⁻¹ 1762 (C═O), 1530 (aromatic); δ_{H} (400 MHz, DMSO-d₆) 2.10 (2H, quaint, J 6.8, 2′-H₂), 3.09 (2H, t, J 6.8, 3′-H₂), 4.28 (2H, t, J 6.8, 1′-H₂), 6.33 (1H, d, J 9.6, 3-H), 6.84 (1H, s, 5′-H), 7.01 (1H, dd, J 8.8, 2.0, 6-H), 7.06 (1H, d, J 2.0, 8-H), 7.66 (1H, d, J 8.8, 5-H), 8.03 (1H, d, J 9.6, 4-H); δ_{C} (100 MHz, DMSO-d₆), 200.9 (C≡O), 162.7 (C═O), 161.3, 156.5, 145.4, 130.6, 113.8, 113.5, 102.3, 98.5, 75.5, 72.5, 68.4, 31.8, 31.0;; Anal. Calc. C, 47.66; H, 2.86. Anal. Found. C, 46.74; H, 2.27.

### Synthesis of 7-(2′,4′,6′-trimethylpyridinium)-4-methyl-2H-chromen-2-one perchlorate salt 14 (MST-220)

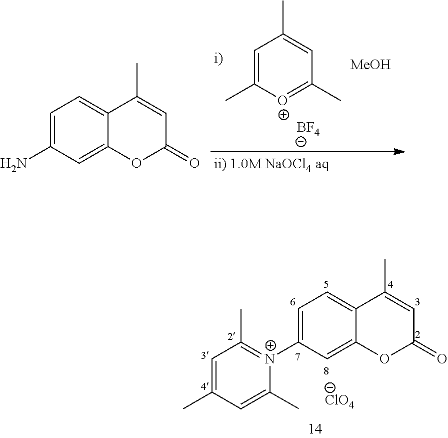

7-Amino-4-methylcoumarin (0.1 g, 1.0 eq) was dissolved in dry MeOH (2.0 ml) and 2,4,6-trimethylpyrilium tetrafluoroborate was added. The mixture was refluxed for 5 h (TLC monitoring) the volume was reduced of 1/3 and the black residue was treated at r.t. with 1.0 M aqueous solution of NaOCl₄ (3.0 eq). The precipitate formed was collected by filtration and crystallized from H₂O to afford the desired product 14 as a white solid.

7-(2′,4′,6′-trimethylpyridinium)-4-methyl-2H-chromen-2-one perchlorate salt 14: 25% yield; Vₘₐₓ (KBr) cm⁻¹ 1760 (C═O), 1540 (aromatic); δ_{H} (400 MHz, DMSO-d₆) 2.39 (6H, s, 2×2′-CH₃), 2.56 (3H, s, 4-CH₃), 2.66 (3H, s, 4′-CH₃), 6.66 (1H, s, 3-H), 7.66 (1H, dd, J 8.4, 2.0, 5-H), 7.85 (1H, d, J 2.0, 8-H), 8.00 (2H, s, 2×3′-H), 8.18 (1H, d, J 8.4, 6-H); be (100 MHz, DMSO-d₆) 160.4, 159.9, 155.6, 154.5, 153.4, 140.9, 122.8, 128.0, 122.7, 122.5, 117.1, 115.9, 22.3, 22.2, 19.0.

### Synthesis of halogenophenylazides

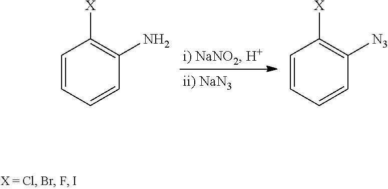

Halogenoaniline (0.3 g, 1.0 eq) was dissolved in a solution H₂O/AcOH (1/2, 10 ml) at 0° C. NaNO₂ (1.4 eq) was slowly added and the resulting solution was stirred at the same temperature for 1 h. Then NaN₃ (1.5 eq) was added portion-wise and the mixture was stirred ar r.t. until starting material was consumed (TLC monitoring). The reaction was quenched with slush, extracted with ethyl acetate (2×20 ml) and the combined organic layers were washed with 5% NaHCO₃ (2×20 ml), dried over Na₂SO₄, filtered off and solvent evaporated in vacuo to afford the corresponding phenylazide which was used without further purification.

### General Procedure for the Synthesis of Click Derivatives

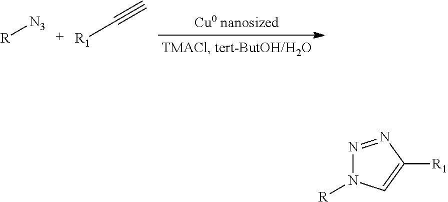

Azide (1.0 eq) and alkine (1.0 eq) were dissolved in tert-ButOH/H₂O 1/1 and then tetramethylamonium chloride (1.0 eq) and copper nanosize (5% mol) were added. The mixture was vigorously stirred at r.t. until starting material was consumed (TLC monitoring). Solvents were removed under vacuo (temperature has not to exceed 40° C.) and the brown residue was purified by silica gel column chromatography eluting with ethyl acetate in n-hexane.

### Synthesis of 7-[(1′H-1′,2′,3′-triazol-4′-yl)methoxy]-2H-chromen-2-one 15 (MST-223)

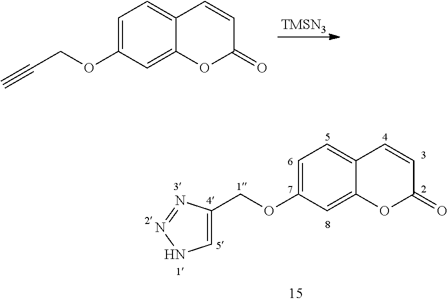

Trimethylsylilazide (0.058 g, 1.0 eq) and 7-(prop-2-ynyloxy)-2H-chromen-2-one (0.1 g, 1.0 eq) were dissolved in tert-ButOH/H₂O 1/1 (2.0 ml) and then tetramethylamonium chloride (0.048 g, 1.0 eq) and copper nanosize (5% mol) were added. The mixture was treated as described and the residue was purified by silica gel column chromatography eluting with 50% ethyl acetate in n-hexane to afford 15 as a white solid.

7-[(1′H-1′,2′,3′-Triazol-4′-yl)methoxy]-2H-chromen-2-one 15: 20% yield; silica gel TLC R_{f} 0.10 (Ethyl Acetate/n-hexane 50% v/v); vₘₐₓ (KBr) cm⁻¹, 1760 (C═O), 1560 (aromatic); δ_{H} (400 MHz, DMSO-d₆) 5.34 (2H, s, 1″-H₂), 7.66 (1H, d, J 9.6, 3-H), 7.06 (1H, dd, J 8.8, 2.4, 6-H), 7.19 (1H, d, J 2.4, 8-H), 7.68 (1H, d, J 8.8, 5-H), 8.03 (1H, d, J 9.6, 4-H), 8.10 (1H, s, 5′-H); be (100 MHz, DMSO-d₆) 162.2, 160.3, 152.4, 144.0, 143.5, 130.0, 129.2, 115.1, 114.2, 112.0, 108.2, 76.2.

### Synthesis of 7-[(1′-(2-bromophenyl)-1H-1′,2′,3′-triazol-4′-yl]methoxy)-2H-chromen-2-one 16 (MST-227)

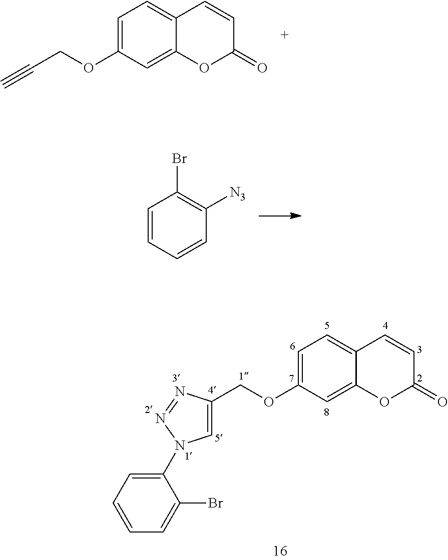 [SMILES: C#CCOc1ccc2ccc(=O)oc2c1 ; [N-]=[N+]=Nc1ccccc1Br ; O=c1ccc2ccc(OCc3cn(-c4ccccc4Br)nn3)cc2o1]

1-Azido-2-bromobenzene (0.44 g, 1.1 eq) and 7-(prop-2-ynyloxy)-2H-chromen-2-one (0.4 g, 1.0 eq) were dissolved in tert-ButOH/H₂O 1/1 (2.0 ml) and then tetramethylamonium chloride (0.4 g, 1.0 eq) and copper nanosize (5% mol) were added. The mixture was treated as described and the residue was purified by silica gel column chromatography eluting with 33% ethyl acetate in n-hexane to afford 16 as a white solid.

7-[(1′-(2-bromophenyl)-1H-1′,2′,3′-triazol-4′-yl]methoxy)-2H-chromen-2-one 16: 50% yield; m.p. 133-134° C.; silica gel TLC R_{f} 0.16 (Ethyl Acetate/n-hexane 33% v/v); vₘₐₓ (KBr) cm⁻¹, 1770 (C═O), 1560 (aromatic); δ_{H} (400 MHz, DMSO-d₆) 5.41 (2H, s, 1″-H₂), 6.35 (1H, d, J 9.6, 3-H), 7.11 (1H, dd, J 8.8, 2.4, 6-H), 7.26 (1H, d, J 2.4, 8-H), 7.67 (4H, m, Ar—H), 7.96 (1H, dd, J 8.8, 2.4, 5-H), 8.04 (1H, d, J 9.6, 4-H), 8.78 (1H, s, 5′-H); be (100 MHz, DMSO-d₆) 162.0, 161.2, 156.2, 145.2, 142.9, 137.0, 134.5, 133.0, 130.5, 129.9, 129.7, 128.1, 119.8, 113.9, 113.7, 113.6, 102.6, 62.4.

### Synthesis of 7-[(1-(2-fluorophenyl)-1H-1,2,3-triazol-4-yl]methoxy)-2H-chromen-2-one 17 (MST-228)

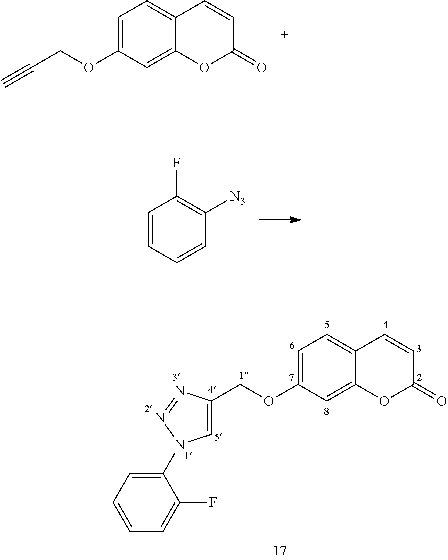 [SMILES: C#CCOc1ccc2ccc(=O)oc2c1 ; [N-]=[N+]=Nc1ccccc1F ; O=c1ccc2ccc(OCc3cn(-c4ccccc4F)nn3)cc2o1]

1-Azido-2-fluorobenzene (0.44 g, 1.1 eq) and 7-(prop-2-ynyloxy)-2H-chromen-2-one (0.4 g, 1.0 eq) were dissolved in tert-ButOH/H₂O 1/1 (2.0 ml) and then tetramethylamonium chloride (0.4 g, 1.0 eq) and copper nanosize (5% mol) were added. The mixture was treated as described and the residue was purified by silica gel column chromatography eluting with 25% ethyl acetate in n-hexane to afford 17 as a pale yellow solid.

7-[(1-(2-Fluorophenyl)-1H-1,2,3-triazol-4-yl]methoxy)-2H-chromen-2-one 17: 30% yield; 161-163° C.; silica gel TLC R_{f} 0.09 (Ethyl Acetate/n-hexane 25% v/v); Vₘₐₓ (KBr) cm⁻¹ 1765 (C═O), 1530 (aromatic); δ_{H} (400 MHz, DMSO-d₆) 5.42 (2H, s, 1″-H₂), 6.35 (1H, d, J 9.6, 3-H), 7.10 (1H, dd, J 8.8, 2.4, 6-H), 7.25 (1H, d, J 2.4, 8-H), 7.50 (1H, m, Ar—H), 7.65 (2H, m, Ar—H), 7.70 (1H, d, J 8.8, 5-H), 7.90 (1H, m, Ar—H), 8.04 (1H, d, J 9.6, 4-H), 8.84 (1H, s, 5′-H); be (100 MHz, DMSO-d₆) 162.0, 161.2, 156.2, 156.0, 153.6, 145.2, 143.6, 132.4, 132.3, 130.5, 127.5, 126.9, 126.5, 118.0, 113.8, 113.7, 102.6, 62.3.

### Synthesis of 6-((1-(2-chlorophenyl)-1H-1,2,3-triazol-4-yl)methoxy)-2H-chromen-2-one 18 (MST-225)

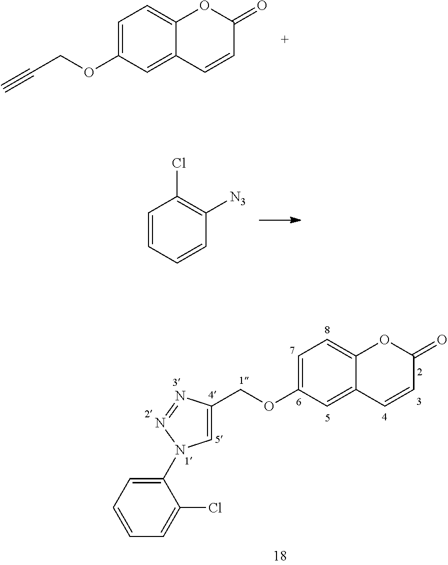 [SMILES: C#CCOc1ccc2oc(=O)ccc2c1 ; [N-]=[N+]=Nc1ccccc1Cl ; O=c1ccc2cc(OCc3cn(-c4ccccc4Cl)nn3)ccc2o1]

1-Azido-2-chlorobenzene (0.44 g, 1.1 eq) and 6-(prop-2-ynyloxy)-2H-chromen-2-one (0.4 g, 1.0 eq) were dissolved in tert-ButOH/H₂O 1/1 (2.0 ml) and then tetramethylamonium chloride (0.4 g, 1.0 eq) and copper nanosize (5% mol) were added. The mixture was treated as described and the residue was purified by silica gel column chromatography eluting with 33% ethyl acetate in n-hexane to afford 18 as a light brown solid.

6-((1-(2-Chlorophenyl)-1H-1,2,3-triazol-4-yl)methoxy)-2H-chromen-2-one 18: 45% yield; m.p. 164-166° C.; silica gel TLC R_{f} 0.10 (Ethyl Acetate/n-hexane 33% v/v); vₘₐₓ (KBr) cm⁻¹ 17562 (C═O), 1520 (aromatic); δ_{H} (400 MHz, DMSO-d₆) 5.35 (2H, s, 1″-H₂), 6.55 (1H, d, J 9.4, 3-H), 7.39 (2H, m, Ar—H, 8-H), 7.51 (1H, d, J 2.4, 5-H), 7.69 (3H, m, Ar—H), 7.82 (1H, dd, J 8.8, 2.4, 7-H), 8.06 (1H, d, J 9.4, 4-H), 8.78 (1H, s, 5′-H); be (100 MHz, DMSO-d₆) 161.0, 155.2, 149.0, 145.0, 144.9, 143.4, 135.3, 132.7, 131.5, 129.5, 129.4, 127.9, 121.0, 120.1, 118.4, 117.6, 113.0, 62.4.

### Synthesis of 6-((1-(2-iodophenyl)-1H-1,2,3-triazol-4-yl)methoxy)-2H-chromen-2-one 19 (MST-226)

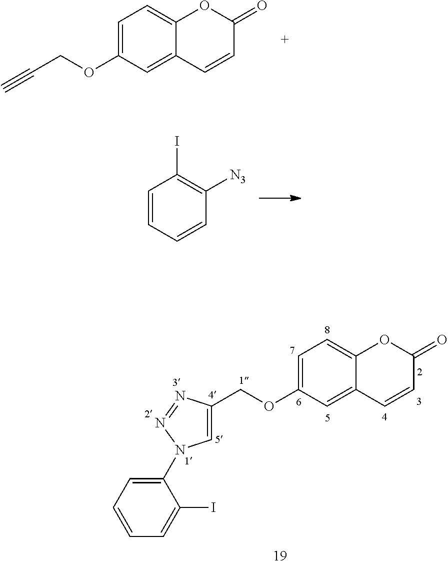 [SMILES: C#CCOc1ccc2oc(=O)ccc2c1 ; [N-]=[N+]=Nc1ccccc1I ; O=c1ccc2cc(OCc3cn(-c4ccccc4I)nn3)ccc2o1]

1-Azido-2-iodobenzene (0.44 g, 1.1 eq) and 6-(prop-2-ynyloxy)-2H-chromen-2-one (0.4 g, 1.0 eq) were dissolved in tert-ButOH/H₂O 1/1 (2.0 ml) and then tetramethylamonium chloride (0.4 g, 1.0 eq) and copper nanosize (5% mol) were added. The mixture was treated as described and the residue was purified by silica gel column chromatography eluting with 33% ethyl acetate in n-hexane to afford 19 as a brown solid.

6-((1-(2-Iodophenyl)-1H-1,2,3-triazol-4-yl)methoxy)-2H-chromen-2-one 19: 30% yield; 162-164° C.; silica gel TLC R_{f} 0.16 (Ethyl Acetate/n-hexane 33% v/v); v_{max (KBr) cm}⁻¹ 1765 (C═O), 1518 (aromatic); δ_{H} (400 MHz, DMSO-d₆) δ_{H} (400 MHz, DMSO-d₆) 5.35 (2H, s, 1″-H₂), 6.54 (1H, d, J 9.4, 3-H), 7.41 (3H, m, Ar—H, 8-H), 7.51 (1H, d, J 2.4, 5-H), 7.64 (2H, m, Ar—H), 8.06 (1H, d, J 9.4, 4-H), 8.14 (1H, dd, J 8.4, 2.4, 7-H), 8.69 (1H, s, 5′-H); be (100 MHz, DMSO-d₆) 161.0, 155.2, 149.0, 145.0, 143.4, 140.7, 140.6, 132.9, 130.3, 129.0, 127.6, 121.1, 120.2, 118.4, 117.6, 113.1, 96.7, 62.6.

### Synthesis of 1-(4-(4-((2-oxo-2H-chromen-6-yloxy)methyl)-1H-1,2,3-triazol-1-yl)-tetrahydro-5-(hydroxymethyl)furan-2-yl)-5-methylpyrimidine-2,4(1H,3H)-dione 20 (MST-221)

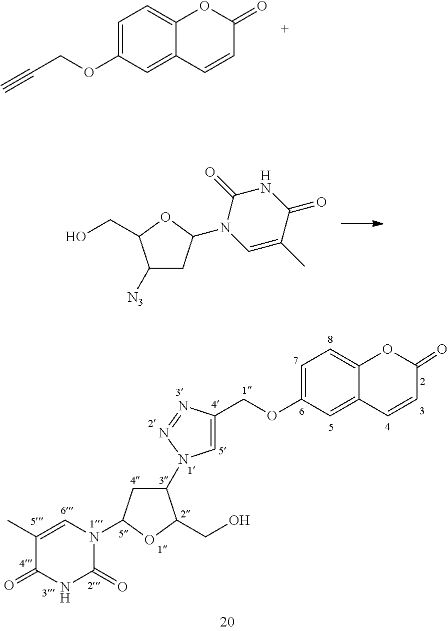 [SMILES: C#CCOc1ccc2oc(=O)ccc2c1 ; Cc1cn(C2CC(N=[N+]=[N-])C(CO)O2)c(=O)[nH]c1=O ; Cc1cn(C2CC(n3cc(COc4ccc5oc(=O)ccc5c4)nn3)C(CO)O2)c(=O)[nH]c1=O]

3′-Azido-3′-deoxythymidine (0.07 g, 1.0 eq) and 6-(prop-2-ynyloxy)-2H-chromen-2-one (0.05 g, 1.0 eq) were dissolved in tert-ButOH/H₂O 1/1 (2.0 ml) and then tetramethylamonium chloride (0.024 g, 1.0 eq) and copper nanosize (5% mol) were added. The mixture was treated as described and the residue was purified by silica gel column chromatography eluting with an increasing amount of ethyl acetate in n-hexane from 50 to 100% to afford 20 as a pale yellow solid.

1′″-(3″-(4′-(2-oxo-2H-chromen-6-yloxy)methyl)-1′H-1′,2′,3′-triazol-1′-yl)-tetrahydro-5-(hydroxymethyl)furan-2-yl)-5′″-methylpyrimidine-2′″,4′″(1′″H,3′″H)-dione 30% yield; silica gel TLC R_{f} 0.21 (Ethyl Acetate 100%); vₘₐₓ (KBr) cm⁻¹, 3150 (O—H), 1760 (C═O), 1525 (aromatic); δ_{H} (400 MHz, DMSO-d₆) 1.85 (3H, s, 5″-CH₃), 2.72 (2H, m, 4″-H₂), 3.70 (2H, m, CH₂OH), 4.26 (1H, m, 2′-H), 5.26 (2H, s, 1″-H₂), 5.32 (1H, t, J 5.6, exchange with D₂O, CH₂OH), 5.43 (1H, m, 3″-H), 6.47 (1H, t, J 6.4, 5″-H), 6.54 (1H, d, J 9.2, 3-H), 7.32 (1H, dd, J 8.8, 2.4, 7-H), 7.35 (1H, d, J 8.8, 8-H), 7.47 (1H, d, J 2.4, 5-H), 7.86 (1H, s, 6′″-H), 8.05 (1H, d, J 9.2, 4-H), 8.49 (1H, s, 5′-H); be (100 MHz, DMSO-d₆) 164.6, 161.0, 155.2, 151.3, 149.0, 144.9, 137.1, 125.3, 120.9, 120.1, 118.3, 117.6, 112.8, 110.5, 85.3, 84.1, 62.6, 61.7, 60.3, 38.1, 30.5, 13.1.

### Synthesis of 6-(tert-butyldimethylsilyloxy)-2H-chromen-2-one (MST-230) and 7-(tert-butyldimethylsilyloxy)-2H-chromen-2-one (MST-231)

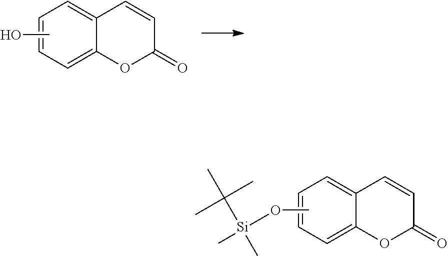

A solution of 6-hydroxy-2H-chromen-2-one or 7-hydroxy-2H-chromen-2-one (0.5 g, 1.0 eq) was treated at r.t. with tert-butyldimethylsilyl chloride (1.1 eq) and Et₃N (1.0 eq) in THF. The reaction was stirred at r.t. until starting material was consumed (TLC monitoring) then quenched with H₂O (40 ml) and extracted with ethyl acetate (3×15 ml). The combined organic layers were washed with H₂O (2×20 ml), dried over Na₂SO₄, filtered-off and concentrated under vacuo to give a residue that was purified by silica gel column cromathography eluting with 20% ethyl acetate/n-hexane v/v.

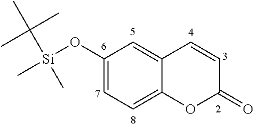 [SMILES: CC(C)(C)[Si](C)(C)Oc1ccc2oc(=O)ccc2c1]

6-(tert-Butyldimethylsilyloxy)-2H-chromen-2-one (MST-230): yield 64% yield; δ_{H} (400 MHz, DMSO-d₆) 0.25 (6H, s, —Si—(CH₃)₂), 1.00 (9H, s, —Si—C(CH₃)₃), 6.51 (1H, d J 9.6, 3-H), 7.13 (1H, dd, J 9.4, 2.4, 7-H), 7.25 (1H, d, J 2.4, 5-H), 7.33 (1H, d, J 9.4, 8-H), 8.00 (1H, d, J 9.6, 4-H); δ_{C} (100 MHz, DMSO-d₆) 161.0 (C═O), 152.2, 149.2, 144.8, 124.9, 120.3, 118.6, 118.3, 117.4, 26.4, 18.8, −3.8.

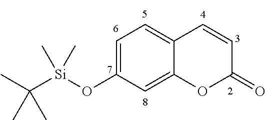 [SMILES: CC(C)(C)[Si](C)(C)Oc1ccc2ccc(=O)oc2c1]

7-(tert-Butyldimethylsilyloxy)-2H-chromen-2-one (MST-231): yield 58% yield; δ_{H} (400 MHz, DMSO-d₆) 0.29 (6H, s, —Si—(CH₃)₂), 1.00 (9H, s, —Si—C(CH₃)₃), 6.34 (1H, d J 9.6, 3-H), 6.88 (1H, dd, J 9.4, 2.4, 6-H), 6.92 (1H, d, J 2.4, 8-H), 7.65 (1H, d, J 9.4, 5-H), 8.04 (1H, d, J 9.6, 4-H); δ_{C} (100 MHz, DMSO-d₆) 162.2 (C═O), 156.4, 145.4, 130.6, 118.1, 114.2, 112.3, 107.9, 103.1, 26.7, 18.7, −2.3.

### Synthesis of 6-(tert-butyldimethylsilyloxy)-2H-chromene-2-thione (MST-232) and 7-(tert-butyldimethylsilyloxy)-2H-chromene-2-thione (MST-234)

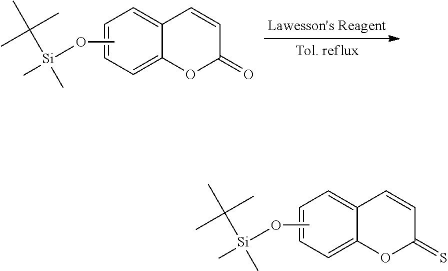

6-(tert-Butyldimethylsilyloxy)-2H-chromen-2-one (MST230) or 7-(tert-butyldimethylsilyloxy)-2H-chromen-2-one (MST-231) (0.5 g, 1.0 eq) was dissolved in dry toluene (20 ml) and treated with Lawesson's reagents (1.5 eq) at reflux for 3 h. The mixture was cooled down to r.t., solvent was removed under vacuo and the residue was partitioned between H₂O and ethyl acetate. The organic layer was washed with H₂O (3×15 ml), dried over Na₂SO₄, filtered and concentrated in vacuo o give a residue that was purified by silica gel column chromatography eluting with 20% ethyl acetate/n-hexane v/v.

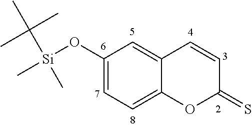 [SMILES: CC(C)(C)[Si](C)(C)Oc1ccc2oc(=S)ccc2c1]

6-(tert-Butyldimethylsilyloxy)-2H-chromene-2-thione (MST-232): yield 60% yield; silica gel TLC R_{f} 0.40 (Ethyl acetate/n-hexane 20% v/v); δ_{H} (400 MHz, DMSO-d₆) 0.27 (6H, s, —Si—(CH₃)₂), 1.01 (9H, s, —Si—C(CH₃)₃), 7.25 (1H, dd J 9.2, 2.8, 7-H), 7.29 (1H, d, J 9.6, 3-H), 7.32 (1H, d, J 2.8, 5-H), 7.55 (1H, d, J 9.2, 8-H), 7.90 (1H, d, J 9.6, 4-H); δ_{C} (100 MHz, DMSO-d₆) 198.0 (C═S), 153.3, 152.2, 136.8, 130.1, 126.0, 122.2, 118.4, 118.3, 26.4, 18.8, −3.8.

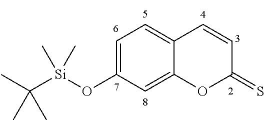 [SMILES: CC(C)(C)[Si](C)(C)Oc1ccc2ccc(=S)oc2c1]

7-(tert-Butyldimethylsilyloxy)-2H-chromene-2-thione (MST-234): yield 61% yield; silica gel TLC R_{f} 0.38 (Ethyl acetate/n-hexane 20% v/v); δ_{H} (400 MHz, DMSO-d₆) 0.31 (6H, s, —Si—(CH₃)₂), 1.00 (9H, s, —Si—C(CH₃)₃), 7.01 (1H, dd J 9.2, 2.8, 6-H), 7.03 (1H, d, J 2.8, 8-H), 7.17 (1H, d, J 9.6, 3-H), 7.76 (1H, d, J 9.2, 5-H), 7.90 (1H, d, J 9.6, 4-H); δ_{C} (100 MHz, DMSO-d₆) 198.2 (C═S), 159.0, 158.2, 137.2, 130.9, 127.5, 126.1, 119.8, 115.9, 26.3, 18.9, −3.8.

### Synthesis of 6-hydroxy-2H-chromene-2-thione (MST-233) and Synthesis of 7-hydroxy-2H-chromene-2-thione (MST-235)

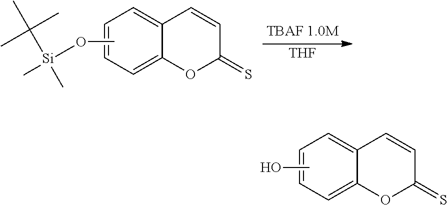

6-(tert-Butyldimethylsilyloxy)-2H-chromene-2-thione (MST-232) or 7-(tert-butyldimethylsilyloxy)-2H-chromene-2-thione (MST-234) (0.3 g, 1.0 eq) was dissolved in THF (2.0 ml) and treated at r.t with TBAF 1.0 M in THF (1.1 eq). The reaction was stirred at r.t. until starting material was consumed (TLC monitoring) and then was quenched with 3.0 M aqueous hydrochloric acid, extracted with ethyl acetate (3×15 ml). The combined organic layers were washed with H₂O (3×20 ml), brine (3×20 ml) dried over Na₂SO₄, filtered, concentrated under vacuo to give a residue that was purified by silica gel column chromatography eluting with 50% ethyl acetate/n-hexane v/v.

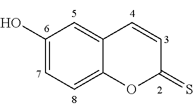 [SMILES: Oc1ccc2oc(=S)ccc2c1]

6-Hydroxy-2H-chromene-2-thione (MST-233): yield 96% yield; silica gel TLC R_{f} 0.35 (Ethyl acetate/n-hexane 50% v/v); δ_{H} (400 MHz, DMSO-d₆) 7.12 (1H, d J 2.8, 5-H), 7.18 (1H, dd, J 9.2, 2.8, 7-H), 7.25 (1H, d, J 9.6, 3-H), 7.50 (1H, d, J 9.2, 8-H), 7.87 (1H, d, J 9.6, 4-H), 10.05 (1H, brs, exchange with D₂O, OH); δ_{C} (100 MHz, DMSO-d₆) 197.8 (C═S), 155.8, 151.1, 137.0, 129.9, 122.1, 121.9, 118.2, 112.9.

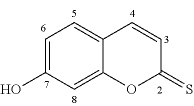 [SMILES: Oc1ccc2ccc(=S)oc2c1]

6-Hydroxy-2H-chromene-2-thione (MST-235): yield 55% yield; silica gel TLC R_{f} 0.40 (Ethyl acetate/n-hexane 50% v/v); δ_{H} (400 MHz, DMSO-d₆) 6.93 (2H, m, 6-H, 8-H), 7.09 (1H, d, J 9.6, 3-H), 7.68 (1H, d, J 9.2, 5-H), 7.85 (1H, d, J 9.6, 4-H), 10.96 (1H, brs, exchange with D₂O, OH); δ_{C} (100 MHz, DMSO-d₆) 198.1 (C═S), 163.3, 159.0, 137.8, 130.9, 126.0, 115.9, 114.1, 102.8.

### Synthesis of allyloxycoumarines

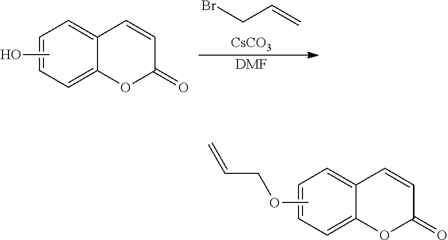

Hydroxycoumarin (1.0 g, 1.0 eq), Cs₂Co₃ (3.0 eq) and allylbromide (3.0 eq) were dissolved in dry DMF (30 ml) and the mixture was stirred at 60° C. O.N. The reaction was quenched with slush and extracted with DCM (3×20 ml). The combined organic layers were washed with brine (3×20 ml), H₂O (5×20 ml), dried over Na₂SO₄, filtered and concentrated under vacuo to give a residue that was crystallized from MeOH/H₂O.

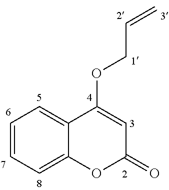

4-(Allyloxy)-2H-chromen-2-one yield (MST-236): 70% yield; δ_{H} (400 MHz, DMSO-d₆) 4.87 (2H, d J 8.0, 1′-H₂), 5.40 (1H, dd, J 13.2, 4.8, 3′-HH), 5.59 (1H, dd, J 15.6, 4.8, 3′-HH), 5.96 (1H, s, 3-H), 6.15 (1H, m, 2′-H), 7.41 (1H, m, 7-H, 8-H), 7.22 (1H, dd, J 8.8, 8.4, 6-H), 7.89 (1H, d, J 8.8, 5-H); be (100 MHz, DMSO-d₆) 165.4 (C═O), 162.5, 153.7, 133.7, 132.6, 125.2, 123.8, 119.7, 117.4, 116.1, 92.0, 70.7.

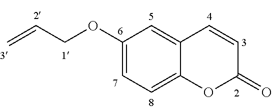

6-(Allyloxy)-2H-chromen-2-one yield (MST-237): 62% yield; δ_{H} (400 MHz, DMSO-d₆) 4.64 (2H, d J 8.0, 1′-H₂), 5.32 (1H, dd, J 13.2, 4.8, 3′-HH), 5.48 (1H, dd, J 15.6, 4.8, 3′-HH), 6.10 (1H, m, 2′-H), 6.16 (1H, d, J 9.6, 3-H), 7.25 (1H, dd, J 9.2, 2.4, 7-H), 7.41 (1H, d, J 2.4, 5-H), 7.36 (1H, d, J 9.2, 8-H), 8.03 (1H, d, J 9.6, 4-H); be (100 MHz, DMSO-d₆) 161.0 (C═O), 155.4, 148.8, 144.9, 134.3, 120.8, 120.1, 118.7, 118.3, 117.5, 112.7, 69.7.

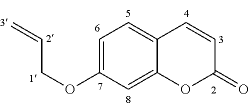

7-(Allyloxy)-2H-chromen-2-one yield (MST-238): 85% yield; δ_{H} (400 MHz, DMSO-d₆) 4.73 (2H, d J 8.0, 1′-H₂), 5.32 (1H, dd, J 13.2, 4.8, 3′-HH), 5.45 (1H, dd, J 15.6, 4.8, 3′-HH), 6.09 (1H, m, 2′-H), 6.33 (1H, d, J 9.6, 3-H), 7.00 (1H, dd, J 9.2, 2.4, 6-H), 7.05 (1H, d, J 2.4, 8-H), 7.67 (1H, d, J 9.2, 5-H), 8.03 (1H, d, J 9.6, 4-H); δ_{C} (100 MHz, DMSO-d₆) 162.0 (C═O), 155.0, 148.3, 144.2, 135.1, 119.2, 118.7, 118.5, 118.0, 117.2, 112.6, 69.5.

### Synthesis of allyloxy-2H-chromene-2-thiones

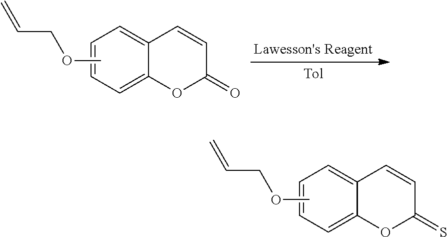

Procedure as for synthesis of (MST-218)

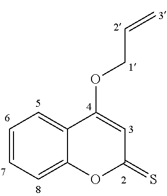

4-(Allyloxy)-2H-chromene-2-thione (MST-239): 61% yield; δ_{H} (400 MHz, DMSO-d₆) 4.95 (2H, d J 6.0, 1′-H₂), 5.42 (1H, dd, J 13.2, 4.8, 3′-HH), 5.59 (1H, dd, J 15.6, 4.8, 3′-HH), 6.15 (1H, m, 2′-H), 6.97 (1H, s, 3-H), 7.51 (1H, t, J 8.8, 7-H), 7.63 (1H, d, J 8.8, 8-H), 7.80 (1H, t, J 8.8, 6-H), 7.96 (1H, d, J 8.8, 5-H); be (100 MHz, DMSO-d₆) 198.5 (C=5), 160.9, 157.2, 134.4, 132.5, 126.6, 123.8, 119.9, 117.5, 117.2, 107.6, 71.2.

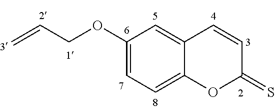

6-(Allyloxy)-2H-chromene-2-thione (MST-240): 62% yield; δ_{H} (400 MHz, DMSO-d₆) 4.68 (2H, d J 8.0, 1′-H₂), 5.32 (1H, dd, J 13.2, 4.8, 3′-HH), 5.49 (1H, dd, J 15.6, 4.8, 3′-HH), 6.09 (1H, m, 2′-H), 7.30 (1H, d, J 9.6, 3-H), 7.36 (1H, dd, J 9.2, 2.4, 7-H), 7.38 (1H, d, J 2.4, 5-H), 7.59 (1H, d, J 9.2, 8-H), 7.89 (1H, d, J 9.6, 4-H); δ_{C} (100 MHz, DMSO-d₆) 197.9 (C═S), 156.3, 151.9, 145.7, 136.8, 134.0, 130.2, 121.9, 118.8, 118.4, 112.1, 69.8.

### Synthesis of 7-(2′-hydroxyethoxy)-2H-chromen-2-one (MST-241)

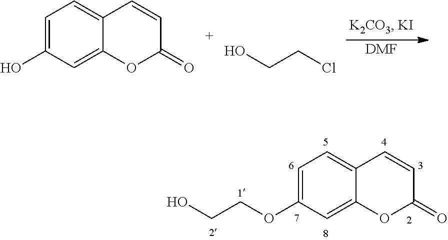

A mixture of 7-hydroxy-2H-chromen-2-one (0.5 g, 1.0 eq), K₂CO₃ (5.0 eq), KI (1.0 eq) and chloroethanol (1.0 eq) in DMF dry (10 ml) was stirred at 60° C. for 5 h. The reaction mixture was cooled down to 0° C., quenched with 6M aqueous hydrochloric acid (50 ml) and extracted with ethyl acetate (3×20 ml). The combined organic layers were washed several timed with H₂O, dried over Na₂SO₄, filtered-off and concentrated under vacuo to afford a residue that was purified by silica gel column chromatography eluting with 50% ethyl acetate/n-hexane v/v.

7-(2′-Hydroxyethoxy)-2H-chromen-2-one (MST-241): 72% yield; silica gel TLC R_{f} 0.10 (Ethyl acetate/n-hexane 50% v/v); δ_{H} (400 MHz, DMSO-d₆) 3.78 (2H, m, 2′-H₂), 4.13 (2H, m, 1′-H₂), 4.97 (1H, t, J 5.6, exchange with D₂O, O—H), 6.30 (1H, d, J 9.6, 3-H), 6.97 (1H, dd, J 9.2, 2.4, 6-H), 7.02 (1H, d, J 2.4, 8-H), 7.65 (1H, d, J 9.2, 5-H), 8.01 (1H, d, J 9.6, 4-H); δ_{C} (100 MHz, DMSO-d₆) 162.8, 161.3, 156.3, 145.3, 130.4, 113.7, 113.4, 102.1, 71.3, 65.9, 60.3.

### Synthesis of 2′-(2-oxo-2H-chromen-7-yloxy)ethyl 4″-methylbenzenesulfonate (MST-242)

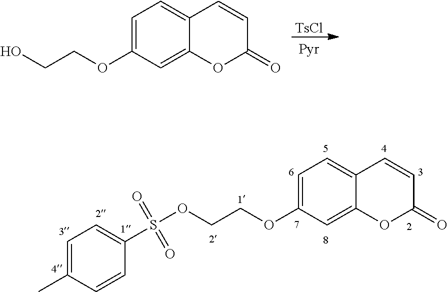 [SMILES: O=c1ccc2ccc(OCCO)cc2o1 ; "TsCl" ; Cc1ccc(S(=O)(=O)OCCOc2ccc3ccc(=O)oc3c2)cc1]

7-(2′-Hydroxyethoxy)-2H-chromen-2-one (MST-241) (0.2 g, 1.0 eq) was dissolved in dry pyridine (5 ml) and treated at 0° C. with TsCl (1.1 eq). The yellow solution was stirred at r.t. until starting material was consumed (TLC monitoring) and then quenched with a 1.0M aqueous hydrochloric acid at 0° C. The mixture was extracted with ethyl acetate (3×15 ml) and the combined organic layers were washed with brine (3×20 ml), H₂O (3×20 ml) dried over Na₂SO₄, filtered-off and concentrated under vacuo to afford a residue that was purified by silica gel column chromatography eluting with 50% ethyl acetate/n-hexane v/v.

2′-(2-Oxo-2H-chromen-7-yloxy)ethyl 4″-methylbenzenesulfonate (MST-242): 35% yield; silica gel TLC R_{f} 0.36 (Ethyl acetate/n-hexane 50% v/v); δ_{H} (400 MHz, DMSO-d₆) 2.44 (3H, s, CH₃). 4.32 (2H, m, 1′-H₂), 4.41 (2H, m, 2′-H₂), 6.34 (1H, d, J 9.6, 3-H), 6.87 (1H, dd, J 9.2, 2.4, 6-H), 6.92 (1H, d, J 2.4, 8-H), 7.49 (2H, d J 8.4, 2×2″-H/3″-H), 7.64 (1H, d, J 9.2, 5-H), 7.83 (2H, d J 8.4, 2×2″-H/3″-H), 8.02 (1H, d, J 9.6, 4-H); δ_{C} (100 MHz, DMSO-d₆) 161.7, 161.1, 156.1, 145.9, 145.1, 133.1, 131.0, 130.4, 128.6, 113.7, 113.6, 113.5, 102.3, 69.7, 66.9, 21.9.

### Synthesis of 7-(2′-fluoroethoxy)-2H-chromen-2-one (MST-243)

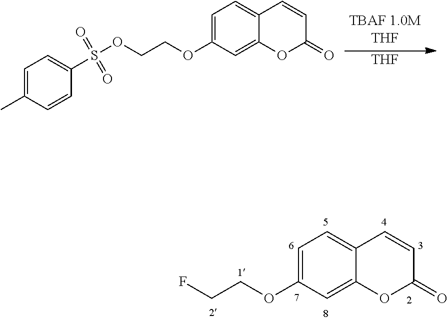 [SMILES: Cc1ccc(S(=O)(=O)OCCOc2ccc3ccc(=O)oc3c2)cc1 ; O=c1ccc2ccc(OCCF)cc2o1 ; "THF"]

2′-(2-Oxo-2H-chromen-7-yloxy)ethyl 4″-methylbenzenesulfonate (MST-242) (0.1 g, 1.0 eq) was dissolved in THF (1.0 ml) and treated with TBAF 1.0M in THF (1.05 eq). The yellow solution was stirred at r.t. for 15 min. Then solvents were removed in vacuo and the residue was purified by silica gel column chromatography eluting with 50% ethyl acetate/n-hexane v/v.

7-(2′-Fluoroethoxy)-2H-chromen-2-one (MST-243): 40% yield; silica gel TLC R_{f} 0.40 (Ethyl acetate/n-hexane 50% v/v); δ_{H} (400 MHz, DMSO-d₆) 4.36 (1H, m, 1′-HH), 4.44 (1H, m, 1′-HH), 4.74 (1H, m, 1′-HH), 4.87 (1H, m, 1′-HH), 6.34 (1H, d, J 9.6, 3-H), 7.02 (1H, dd, J 9.2, 2.4, 6-H), 7.09 (1H, d, J 2.4, 8-H), 7.68 (1H, d, J 9.2, 5-H), 8.03 (1H, d, J 9.6, 4-H); be (100 MHz, DMSO-d₆) 162.2, 161.1, 145.2, 130.5, 113.63, 113.60, 113.5, 102.3, 82.0 (d, ¹J_{C-F} 166, C-2′), 68.6 (d, ²J_{C-F} 18, C-1′), δ_{F} (376 MHz, DMSO-d₆) −222.23 (1F, s).

### Synthesis of N-(4-methyl-2-oxo-2H-chromen-7-yl)acetamide (MST-244)

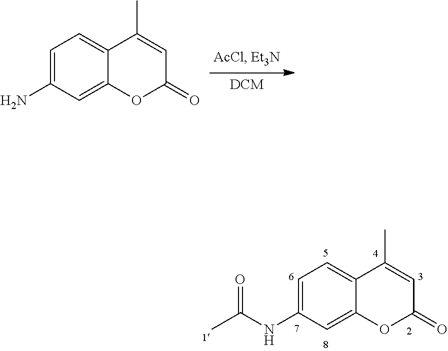 [SMILES: Cc1cc(=O)oc2cc(N)ccc12 ; CC(=O)Nc1ccc2c(C)cc(=O)oc2c1]

A suspension of 7-amino-4-methyl-2H-chromen-2-one (0.1 g, 1.0 eq) in DCM dry (5.0 ml) was treated with acetyl chloride (1.0 eq) and Et₃N (1.0 eq) under reflux for 7 h. Solvents were removed under vacuo and the residue was purified by silica gel column chromatography eluting with 50% ethyl acetate/n-hexane v/v.

N-(4-Methyl-2-oxo-2H-chromen-7-yl)acetamide (MST-244): 73% yield; silica gel TLC R_{f} 0.11 (Ethyl acetate/n-hexane 50% v/v); δ_{H} (400 MHz, DMSO-d₆) 2.14 (3H, s, 1′-CH₃), 2.43 (3H, s, 4-CH₃), 6.29 (1H, s, 3-H), 7.50 (1H, dd, J 9.2, 2.4, 6-H), 7.74 (1H, d, J 9.2, 5-H), 7.79 (1H, d, J 2.4, 8-H), 10.40 (1H, brs, exchange with D₂O, N—H); δₑ (100 MHz, DMSO-d₆) 170.0, 161.0, 154.6, 154.0, 143.5, 126.8, 115.9, 115.7, 113.0, 106.3, 25.1, 18.9.

### Synthesis of 1-(3′,5′-dimethylphenyl)-3-(4-methyl-2-oxo-2H-chromen-7-yl)urea (MST-245)

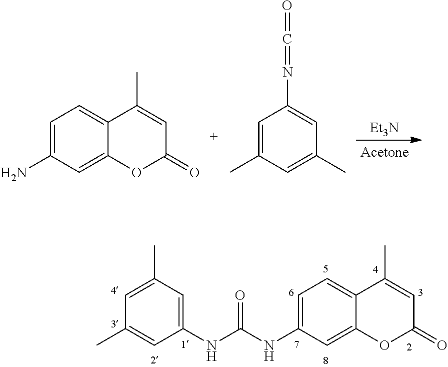

7-amino-4-methyl-2H-chromen-2-one (0.1 g, 1.0 eq) in acetone (10 ml) was treated at reflux with 3,5-dimethyyl isocyanate (1.0 eq) and Et₃N (1.1 eq) for 24 h. Then the solvents were removed in vacuo and the residue was purified by silica gel column chromatography eluting with 50% ethyl acetate/n-hexane v/v.

1-(3′,5′-Dimethylphenyl)-3-(4-methyl-2-oxo-2H-chromen-7-yl)urea (MST-245): 23% yield; silica gel TLC R_{f} 0.22 (Ethyl acetate/n-hexane 50% v/v); δ_{H} (400 MHz, DMSO-d₆) 2.28 (6H, s, 2×3′-CH₃), 2.43 (3H, s, 4-CH₃), 6.25 (1H, s, 3-H), 6.69 (1H, s, 4′-H), 7.13 (2H, s, 2×2′-H), 7.39 (1H, dd, J 9.2, 2.4, 6-H), 7.65 (1H, d, J 2.4, 8-H), 7.71 (1H, d, J 9.2, 5-H), 8.72 (1H, s, exchange with D₂O, N—H), 9.20 (1H, s, exchange with D₂O, N—H); δ_{C} (100 MHz, DMSO-d₆) 161.1, 254.2, 153.4, 144.5, 140.6, 140.0, 138.9, 138.6, 126.9, 124.9, 124.3, 117.3, 116.8, 115.3, 22.1, 19.0.

### Synthesis of tert-butyl 4-methyl-2-oxo-2H-chromen-7-ylcarbamate (MST-246)

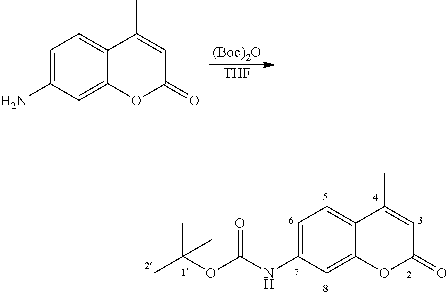 [SMILES: "(Boc)2O" ; Cc1cc(=O)oc2cc(N)ccc12 ; Cc1cc(=O)oc2cc(NC(=O)OC(C)(C)C)ccc12 ; "THF"]

A suspension of 7-amino-4-methyl-2H-chromen-2-one (0.1 g, 1.0 eq) in THF dry (2.0 ml) was treated at reflux with di-tert-butyl dicarbonate (1.0 eq) and Et₃N (1.1 eq) for 24 h. Then the solvents were removed in vacuo and the residue was purified by silica gel column chromatography eluting with 50% ethyl acetate/n-hexane v/v.

tert-Butyl 4-methyl-2-oxo-2H-chromen-7-ylcarbamate (MST-246): 28% yield; silica gel TLC R_{f} 0.42 (Ethyl acetate/n-hexane 50% v/v); δ_{H} (400 MHz, DMSO-d₆) 1.54 (9H, s, 3×2′-CH₃), 2.42 (3H, s, 4-CH₃), 6.26 (1H, s, 3-H), 7.44 (1H, dd, J 9.2, 2.4, 6-H), 7.57 (1H, d, J 2.4, 8-H), 7.70 (1H, d, J 9.2, 5-H), 9.92 (1H, s, exchange with D₂O, N—H); δ_{C} (100 MHz, DMSO-d₆) 161.0, 154.8, 154.2, 153.4, 144.1, 126.8, 115.1. 113.0, 105.2. 80.9, 28.9, 27.8, 18.9.

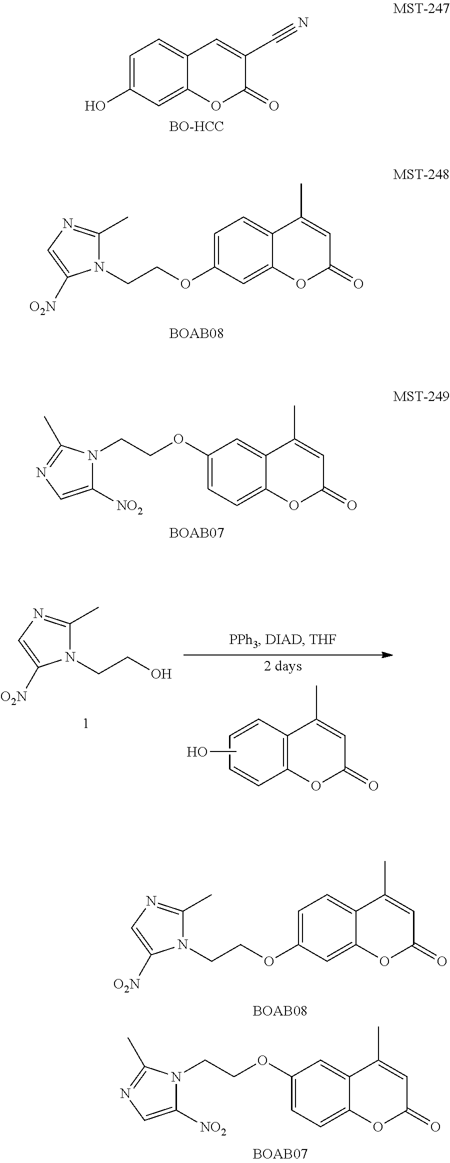

Metronidazole (1 equiv.), 6- or 7-hydroxy-4-methyl coumarine (1 equiv.), and triphenylphospine (1.2 equiv.) are mixed in THF and then diisopropyl azidocarboxylate (DIAD), (1.2 equiv.) is added dropwise. The reaction is stirred 2 days at room temperature. The precipitate is then filtered, washed two times with cold THF and dried under vacuum.

### 7-O-[2-(2-methyl-5-nitro-imidazol-1-yl)ethyl]-4-methylcoumarine (MST-248)

Yield 48%; Rf: 0.11 (AcOEt 8/Et₂O 2); Mp: 238-240° C.; ¹H NMR (400 MHz, DMSO): δ ppm 1.55 (s, 3H), 1.69 (s, 3H), 3.63 (t, 2H, J=5.00 Hz), 3.91 (t, 2H, J=5.00 Hz), 5.38 (d, 1H, J=1.05 Hz), 6.07 (dd, 1H, J=2.49 Hz, J=8.81 Hz), 6.15 (d, 1H, J=2.49 Hz), 6.84 (d, 1H, J=8.81 Hz), 7.20 (s, 1H); ¹³C NMR (101 MHz, DMSO): δ ppm 14.10, 18.08, 45.00, 67.02, 101.28, 111.38, 112.27, 113.45, 126.54, 132.87, 151.71, 153.25, 154.56, 160.00, 160.66. MS ESI⁺/ESI⁻: m/z 330.34 (M+H)⁺, 328.38 (M−H)⁻.

### 6-O-[2-(2-methyl-5-nitro-imidazol-1-yl)ethyl]-4-methylcoumarine (MST-249)

Yield 42%; Rf: 0.16 (AcOEt 8/Et₂O 2); Mp: 190-191° C.; ¹H NMR (400 MHz, DMSO): δ ppm 1.56 (s, 3H), 1.70 (s, 3H), 3.57 (t, 2H, J=5.00 Hz), 3.89 (t, 2H, J=5.00 Hz), 5.53 (s, 1H); 6.32 (m, 2H), 6.46 (d, 1H, J=9.70 Hz), 7.19 (s, 1H); ¹³C NMR (101 MHz, DMSO): δ ppm 14.15, 18.13, 45.19, 66.97, 108.61, 114.72, 117.58, 120.07, 132.93, 138.31, 147.47, 151.83, 152.94, 154.06, 159.76; MS ESI⁺/ESI⁻: m/z 330.34 (M+H)⁺, 328.38 (M−H)⁻.

### Example 4

### In Vitro CA Inhibition Assays Data

The methods for achieving this data are shown, for example, in Maresca, A. et al, J. Med. Chem. 2010, 53,335-344.

TABLE 1
hCA I, II, IX and XII inhibition data in K_{I} (μM)

| Compound | hCA I | hCA II | hCA IX | | hCA XII | |
|---|---|---|---|---|---|---|
| MST-201 | 3.4 | >100 | 0.35 | | 105 | nM |
| MST-202 | 1.0 | >100 | 0.37 | | 54 | nM |
| MST-203 | 0.59 | 77 | 93 | nM | 8.5 | nM |
| MST-204 | >100 | >100 | 9.2 | nM | 43 | nM |
| MST-205 | >100 | >100 | 201 | nM | 184 | nM |
| MST-206 | 0.88 | 0.59 | 0.82 | | 101 | nM |
| MST-207 | >100 | >100 | 3.2 | | 53 | nM |
| MST-208 | 15400 | >100 000 | 56.0 | | 25.2 | |
| MST-209 | 16200 | >100 000 | 400 | | 52.4 | |
| MST-210 | 570 | 21500 | 0.97 | | 26.5 | |
| MST-211 | 730 | 43300 | 6.7 | | 95.2 | |
| MST-212 | 850 | 92000 | 19.6 | | 96.0 | |
| MST-213 | 8450 | >100 000 | 63.8 | | 48.3 | |
| MST-214 | 7015 | >100 000 | 52.3 | | 61.2 | |
| MST-215 | 6970 | >100 000 | 47.9 | | 45.6 | |
| MST-216 | 7625 | >100 000 | 47.3 | | 30.2 | |
| MST-217 | 950 | >100 000 | 41.6 | | 38.9 | |
| MST-218 | 960 | >100 000 | 47.5 | | 24.7 | |
| MST-219 | 1170 | >100 000 | 66.1 | | 41.4 | |
| MST-220 | 52900 | >100 000 | 8.8 | | 61.6 | |
| MST-221 | 87.8 | 913 | 9.3 | | 49.9 | |
| MST-222 | 15400 | >100 000 | 8.6 | | 46.5 | |
| MST-223 | >100 000 | >100 000 | 9.1 | | 70.4 | |
| MST-224 | 85.8 | >100 000 | 61.2 | | 31.0 | |
| MST-225 | >100 000 | >100 000 | 7.5 | | 48.6 | |
| MST-226 | 57.6 | >100 000 | 6.4 | | 40.8 | |
| MST-227 | 40.6 | >100 000 | 6.8 | | 27.0 | |
| MST-228 | 117 | >100 000 | 7.5 | | 18.7 | |
| MST-229 | >100 | >100 | 0.56 | | 8.10 | |
| MST-230 | 8.78 | >200 | 0.80 | | 0.28 | |
| MST-231 | 8.32 | >200 | 0.85 | | 0.83 | |
| MST-232 | 7.57 | >200 | 0.86 | | 0.31 | |
| MST-233 | 7.17 | >200 | 0.80 | | 0.34 | |
| MST-234 | 8.18 | >200 | 0.96 | | 0.35 | |
| MST-235 | 8.02 | >200 | 0.78 | | 0.32 | |
| MST-239 | 8.51 | >200 | 3.26 | | 1.25 | |
| MST-240 | 7.60 | >200 | 3.23 | | 2.83 | |
| MST-247 | 104 | >200 | 0.24 | | 7.11 | |
| MST-248 | >200 | >200 | 0.37 | | 0.39 | |
| MST-249 | >200 | >200 | 0.40 | | 53 | |

MST-229 = 7-hydroxy-4-methylcoumarin, available from chemical vendors

### Example 5

For the IN VIVO examples, the following methods and additional information will be a useful reference.

### Pharmacological Inhibitors

For in vivo studies, the inhibitors were administered by intraperitoneal injection. The compounds were solubilized in 37.5% PEG400/12.5% ethanol/50% saline prior to injection. Inhibitor concentrations ranged from 4.5 mM to 12 mM. The exact concentrations used were dependent on the upper limit of solubility of a particular inhibitor in the PEG400/ethanol/saline vehicle. Inhibitor concentrations were converted to mg/kg for in vivo administration and are reported as such in the examples. Conversion to mg/kg was based on a 200 μl injection volume for a 20 g mouse. Vehicle components were held constant as inhibitor concentrations were varied. Inhibitors were administered daily for 5-6 days and images were acquired 24 hours following the final dose.

### Tumorigenesis and Spontaneous Metastasis Analysis

All animal procedures were done in accordance with protocols approved by the Institution Animal Care Committee at the BC Cancer Research Centre and The University of British Columbia (Vancouver, BC, Canada). Progression of metastases was monitored and quantified using non-invasive in vivo bioluminescent imaging (IVIS) as previously described (Lou, Y., Preobrazhenska, O., auf dem Keller, U., et al. (2008) Dev Dyn 237: 2755-2768). Mice were monitored daily and moribund animals were sacrificed in accordance with ethical guidelines. For studies involving experimental lung metastasis, mice were injected intravenously through the tail vein with 2×10⁵ cells per animal. Mice were imaged once per week to follow the establishment and growth of lung metastases. Mice were euthanized by 20 days post-injection. Tumor burden in the lung was quantified using bioluminescence data acquired by imaging with IVIS.

### Statistical Analysis

Results were subjected to statistical analysis using the Data Analysis ToolPack™ in Excel software. Two-tailed p values were calculated using Student's t-test. Data were considered significant for p<0.05.

In Vivo Metastases Inhibition with Novel Glycosylcoumarin MST-204

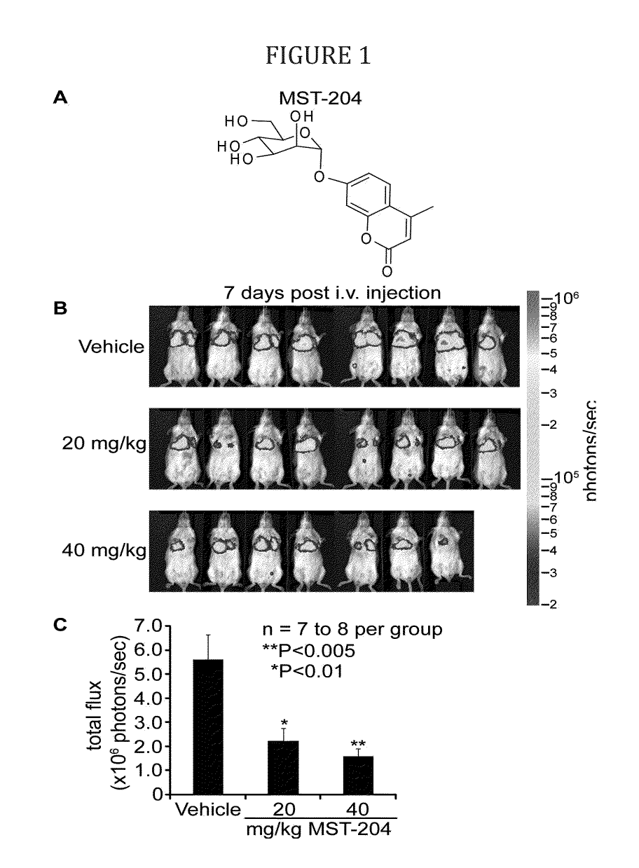

4T1 cells injected intravenously form robust lung metastases and subject mice have to be euthanized within 3 weeks post injection due to metastatic progression. Novel CAIX inhibitor MST-204 reduced the formation of metastases by 4T1 mammary tumor cells. In FIG. 1, the Chemical structure of CAIX inhibitor MST-204 is shown. Representative bioluminescent images of metastases established following intravenous injection of 2×10⁵ 4T1 cells per mouse and treatment with MST-204 are shown in FIG. 1B. Animals were treated 24 hours post inoculation of cells. The inhibitor was administered daily by i.p. injection for 6 days and the mice were imaged 24 hours following the final dose of inhibitor. MST-204 was delivered in a vehicle comprised of 37.5% PEG400, 12.5% ethanol and 50% saline. Mice dosed with vehicle alone served as controls. As for quantification of tumor-derived bioluminescence, as shown in FIG. 1C regions of interest were positioned around metastatic foci and total flux (photons/sec) at the mouse surface was calculated. Data are reported as the mean±s.e.m. N=7-8 per group. *P<0.01. **P<0.005.

In Vivo Metastases Inhibition with Novel Glycosylcoumarin MST-205

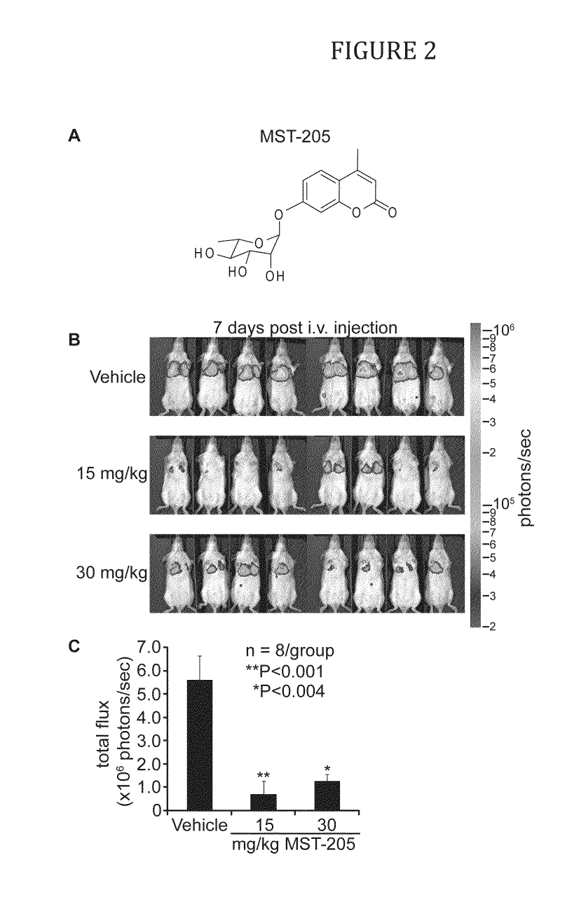

MST-205 inhibits the formation of metastases by 4T1 mammary tumor cells. Animals were treated 24 hours post inoculation of cells. The inhibitor was administered daily by i.p. injection for 6 days and the mice were imaged 24 hours following the final dose of inhibitor. MST-205 was delivered in a vehicle comprised of 37.5% PEG400, 12.5% ethanol and 50% saline. Mice dosed with vehicle alone served as controls. Representative bioluminescent images of metastases established following intravenous injection of 2×10⁵ 4T1 cells and treatment with MST-205 (FIG. 2B). In images of tumor-derived bioluminescence, regions of interest were positioned around metastatic foci and total flux (photons/sec) at the mouse surface was calculated. Data are reported as the mean±s.e.m, and shown in the graph in FIG. 2C. N=8 per group. *P<0.004. **P<0.001.

### MST-205 Attenuates the Growth of 4T1 Primary Tumors

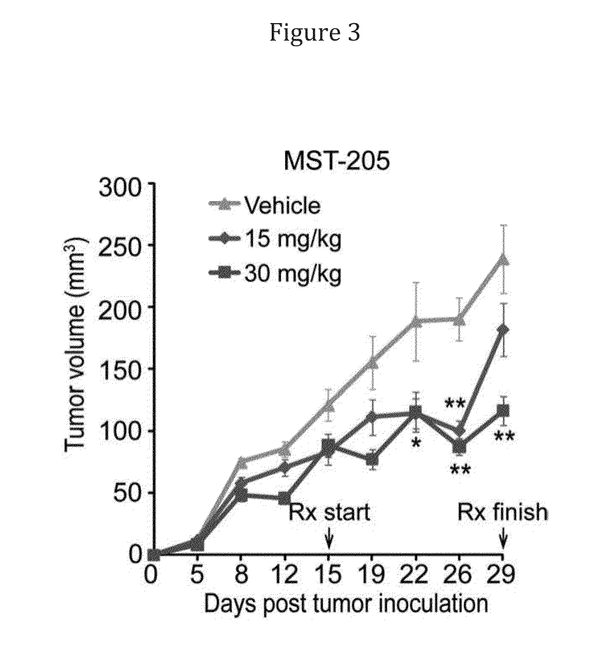

4T1 cells (1×10⁶ cells/mouse) were orthotopically implanted into female BALB/c mice and tumors were allowed to establish for 14 days. Animals then received MST-205 daily by i.p. injection for 14 days. MST-205 was delivered in a vehicle comprised of 37.5% PEG400, 12.5% ethanol and 50% saline. Tumor growth was monitored 2 times per week by caliper-based measurement. Treatment initiation and termination are indicated by arrows. Vehicle-treated animals served as controls. n=8 for each group. *P<0.01, **P<0.003, compared to vehicle controls. Results are shown in FIG. 3.

While specific embodiments of the invention have been described and illustrated, such embodiments should be considered illustrative of the invention only and not as limiting the invention as construed in accordance with the accompanying

### REFERENCES

1. Maresca, A.; Temperini, C.; Vu, H.; Pham, N. B.; Poulsen, S. A.; Scozzafava, A.; Quinn, R. J.; Supuran, C. T. Non-zinc mediated inhibition of carbonic anhydrases: coumarins are a new class of suicide inhibitors. J. Am. Chem. Soc. 2009, 131, 3057-3062.
2. Supuran, C. T. Carbonic anhydrases: novel therapeutic applications for inhibitors and activators. Nat. Rev. Drug Discov. 2008, 7, 168-181.
3. Vu, H.; Pham, N. B.; Quinn, R. J. Direct screening of natural product extracts using mass spectrometry. J. Biomol. Screen. 2008, 13, 265-275.
4. a) Supuran, C. T. Carbonic anhydrases as drug targets—general presentation. In Drug Design of Zinc-Enzyme Inhibitors: Functional, Structural, and Disease Applications, Supuran, C. T.; Winum, J. Y. Eds., Wiley, Hoboken (NJ), 2009, pp. 15-38; b) Winum, J. Y.; Rami, M.; Scozzafava, A.; Montero, J. L.; Supuran, C. Carbonic Anhydrase IX: a new druggable target for the design of antitumor agents. Med. Res. Rev. 2008, 28, 445-463; c) Supuran, C. T.; Scozzafava, A.; Casini, A. Carbonic anhydrase inhibitors. Med. Res. Rev. 2003, 23, 146-189.
5. a) Alterio, V.; Di Fiore, A.; D'Ambrosio, K.; Supuran, C. T.; De Simone, G. X-Ray crystallography of CA inhibitors and its importance in drug design. In Drug Design of Zinc-Enzyme Inhibitors: Functional, Structural, and Disease Applications, Supuran, C. T.; Winum, J. Y. Eds., Wiley, Hoboken, 2009, pp. 73-138; b) Mincione, F.: Scozzafava, A.; Supuran, C. T. Antiglaucoma carbonic anhydrase inhibitors as ophthalomologic drugs. In Drug Design of Zinc-Enzyme Inhibitors: Functional, Structural, and Disease Applications, Supuran, C. T.; Winum, J. Y. Eds., Wiley, Hoboken (NJ), 2009, pp. 139-154.
6. a) Köhler, K.; Hillebrecht, A.; Schulze Wischeler, J.; Innocenti, A.; Heine, A.; Supuran, C. T.; Klebe, G. Saccharin inhibits carbonic anhydrases: Possible explanation for its unpleasant metallic aftertaste. Angew. Chem. Int. Ed. Engl. 2007, 46, 7697-7699; b) Alterio, V.; Vitale, R. M.; Monti, S. M.; Pedone, C.; Scozzafava, A.; Cecchi, A.; De Simone, G.; Supuran, C. T. Carbonic anhydrase inhibitors: X-ray and molecular modeling study for the interaction of a fluorescent antitumor sulfonamide with isozyme II and IX. J. Am. Chem. Soc. 2006, 128, 8329-8335.
7. Ebbesen, P.; Pettersen, E. O.; Gorr, T. A.; Jobst, G.; Williams, K.; Kienninger, J.; Wenger, R. H.; Pastorekova, S.; Dubois, L.; Lambin, P.; Wouters, B. G.; Supuran, C. T.; Poellinger, L.; Ratcliffe, P.; Kanopka, A.; Gorlach, A.; Gasmann, M.; Harris, A. L.; Maxwell, P.; Scozzafava, A. Taking advantage of tumor cell adaptations to hypoxia for developing new tumor markers and treatment strategies. J. Enzyme Inhib. Med. Chem. 2009, 24 (S1), 1-39.
8. Schlicker, C.; Hall, R. A.; Vullo, D.; Middelhaufe, S.; Gertz, M.; Supuran, C. T.; Muhlschlegel, F. A.; Steegborn, C. Structure and inhibition of the CO₂-sensing carbonic anhydrase Can2 from the pathogenic fungus Cryptococcus neoformans. J. Mol. Biol. 2009, 385, 1207-1220.
9. a) Thiry, A.; Dogné, J. M.; Masereel, B.; Supuran, C. T. Targeting tumor-associated carbonic anhydrase IX in cancer therapy. Trends Pharmacol. Sci. 2006, 27, 566-573;
9.b) Svastova, E.; Hulikova, A.; Rafajova, M.; Zatovicova, M.; Gibadulinova, A.; Casini, A.; Cecchi, A.; Scozzafava, A.; Supuran, C. T.; Pastorek, J.; Pastorekova, S. Hypoxia activates the capacity of tumor-associated carbonic anhydrase IX to acidify extracellular pH. FEBS Lett. 2004, 577, 439-445.
10. a) Supuran, C. T. Diuretics: From classical carbonic anhydrase inhibitors to novel applications of the sulfonamides. Curr. Pharm. Des. 2008, 14, 641-648;
10.b) Supuran, C. T.; Di Fiore, A.; De Simone, G. Carbonic anhydrase inhibitors as emerging drugs for the treatment of obesity. Expert Opin. Emerg. Drugs. 2008, 13, 383-392;
11. a) Minakuchi, T.; Nishimori, I.; Vullo, D.; Scozzafava, A.; Supuran, C. T. Molecular cloning, characterization and inhibition studies of the Rv1284 β-carbonic anhydrase from Mycobacterium tuberculosis with sulfonamides and a sulfamate. J. Med. Chem. 2009, 52, 2226-2232;
11.b) Nishimori, I.; Minakuchi, T.; Vullo, D.; Scozzafava, A.; Innocenti, A.; Supuran, C. T. Carbonic anhydrase inhibitors. Cloning, characterization and inhibition studies of a new β-carbonic anhydrase from Mycobacterium tuberculosis. J. Med. Chem. 2009, 52, 3116-3120.
12. S. M. Sethna, N. M. Shah The chemistry of coumarins. Chem. Rev. 1945, 36, 1-62.
13. Maresca A, Supuran C T. Coumarins incorporating hydroxy- and chloro-moieties selectively inhibit the transmembrane, tumor-associated carbonic anhydrase isoforms IX and XII over the cytosolic ones I and II. Bioorg Med Chem. Lett. 2010 Aug. 1; 20(15):4511-4.
14. Winum, J-Y., Poulsen, S. A.; Supuran C. T. Therapeutic applications of glycosidic carbonic anhydrase inhibitors. Med. Res. Rev. 2009, 29, 419-35.
15. Maresca, A; Temperini, C; Pochet, L; Masereel, B; Scozzafava, A; and Supuran, C. Deciphering the mechansim of carbonhic anhydrase inhibition with coumarins an thiocoumarins. J Med Chem 2010, 53, 335-344.

1.-30. (canceled)

31. A method for treating a mammal having hypoxic or metastatic cancer, the method comprising administering to the mammal a pharmaceutical composition in an amount that is capable of inhibiting human carbonic anhydrase IX and XII while leaving carbonic anhydrase I and II substantially unaffected, wherein the pharmaceutical composition comprises a compound of Formula V and a pharmaceutically acceptable excipient: 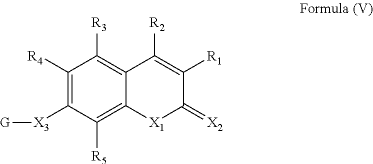
wherein,
G is (1) a glycosyl group, (2) a non-glycosyl group selected from the group of H, alkyl, alkenyl, alkynyl, cycloalkyl, heterocyclic and aryl or (3) a heterocyclic sugar according to the following general formula: 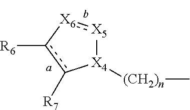
a is a single bond, or a double bond;
b is a single bond, or a double bond;
X₁ and X₂ are independently O or S;
X₃ is —O—, —NH—, —S—, or a single bond;
X₄ is —N—, or —C—;
X₅ are X₆ are independently —N—, —C—, or —O—;
n=0, or 1.
R₁═H; and
R₂; R₃; R₄; R₅; R₆ and R₇ are independently H, alkyl, alkenyl, alkynyl, cycloalkyl, heterocyclic, aryl, or halogen.

32. The method of claim 31 wherein X₁, X₂ and X₃ are all O.

33. The method of claim 31 wherein R₂ is CH₃.

34. The method of claim 31, wherein R₃, R₄ and R₅ are each hydrogen.

35. The method of claim 31 selected from the group consisting of:

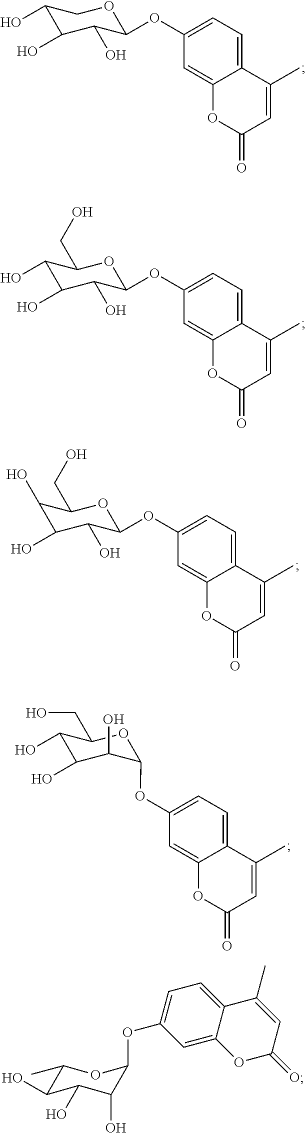

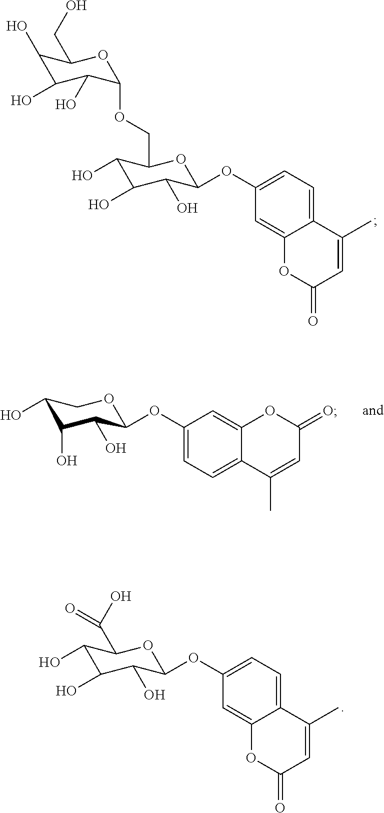

36. A method for treating hypoxic or metastatic cancer in a subject in need thereof comprising administering to the subject a pharmaceutical composition in an amount that is capable of inhibiting human carbonic anhydrase IX and XII while leaving carbonic anhydrase I and II substantially unaffected, wherein the pharmaceutical composition comprises a pharmaceutically acceptable excipient and a compound or a salt thereof selected from the following group:
2′,4′,6′-trimethyl-1-(2-(2-oxo-2H-chromen-7-yloxy)ethyl)pyridine;
7-(pent-4-ynyloxy)-2H-chromen-2-one hexacarbonyldicobalt;
7-(prop-2-ynyloxy)-2H-chromene-2-thione hexacarbonyldicobalt;
7-(prop-2-ynyloxy)-2H-chromen-2-one hexacarbonyldicobalt;
N-(4-methyl-2-oxo-2H-chromen-7-yl)-4′-methylbenezenesulfonimde;
7-(prop-2-ynyloxy)-2H-chromene-2-thione;
7-(allyloxy)-2H-chromene-2-thione;
2-(2-oxo-2H-chromen-7-yloxy)ethylcarbamate;
1-(4-(4-((2-oxo-2H-chromen-6-yloxy)methyl)-1H-1,2,3-triazol-1-yl)-tetrahydro-5-(hydroxymethyl)furan-2-yl)-5-methylpyrimidine-2,4(1H,3H)dione;
7-[(1′H-1′,2′,3′-triazol-4′-yl)methoxy]-2H-chromen-2-one;
6-((1-(2-chlorophenyl)-1H-1,2,3-triazol-4-yl)methoxy)-2H-chromen-2-one
6-((1-(2-bromophenyl)-1H-1,2,3-triazol-4-yl)methoxy)-2H-5-chromen-2-one;
6-((1-(2-fluorophenyl)-1H-1,2,3-triazol-4-yl)methoxy)-2H-5-chromen-2-one;
6-((1-(2-iodophenyl)-1H-1,2,3-triazol-4-yl)methoxy)-2H-5-chromen-2-one;
7-(2′,4′,6′-trimethylpyridinium)-4-methyl-2H-chromen-2-one;
7-(2′,4′,6′-trimethylpyridinium)-4-methyl-2H-chromen-2-one perchlorate;
4-methyl-7-(4-((2-oxo-2H-chromen-7-yloxy)methyl)-1H-1,2,3-triazol-1-yl)-2H-chromen-2-one; 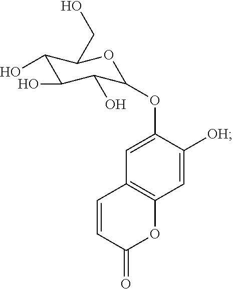 [SMILES: O=c1ccc2cc(OC3OC(CO)C(O)C(O)C3O)c(O)cc2o1] 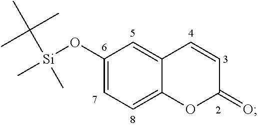 [SMILES: CC(C)(C)[Si](C)(C)Oc1ccc2oc(=O)ccc2c1]
6-(tert-Butyldimethylsilyloxy)-2H-chromen-2-one; 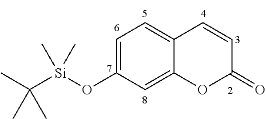 [SMILES: CC(C)(C)[Si](C)(C)Oc1ccc2ccc(=O)oc2c1]
7-(tert-Butyldimethylsilyloxy)-2H-chromen-2-one; 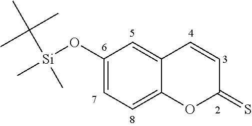 [SMILES: CC(C)(C)[Si](C)(C)Oc1ccc2oc(=S)ccc2c1]
6-(tert-Butyldimethylsilyloxy)-2H-chromene-2-thione; 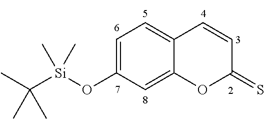 [SMILES: CC(C)(C)[Si](C)(C)Oc1ccc2ccc(=S)oc2c1]
7-(tert-Butyldimethylsilyloxy)-2H-chromene-2-thione; 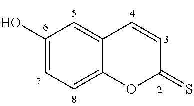 [SMILES: Oc1ccc2oc(=S)ccc2c1]
6-Hydroxy-2H-chromene-2-thione; 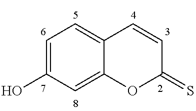 [SMILES: Oc1ccc2ccc(=S)oc2c1]
6-Hydroxy-2H-chromene-2-thione; 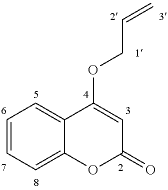
4-(Allyloxy)-2H-chromen-2-one; 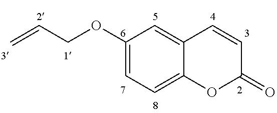
6-(Allyloxy)-2H-chromen-2-one; 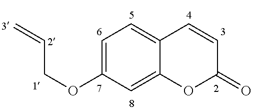
7-(Allyloxy)-2H-chromen-2-one; 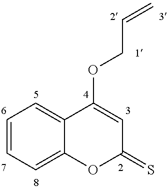
4-(Allyloxy)-2H-chromene-2-thione; 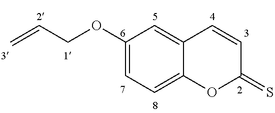
6-(Allyloxy)-2H-chromene-2-thione; 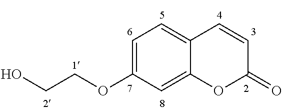 [SMILES: O=c1ccc2ccc(OCCO)cc2o1]
7-(2′-hydroxyethoxy)-2H-chromen-2-one; 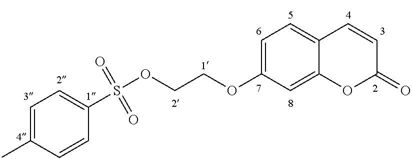 [SMILES: Cc1ccc(S(=O)(=O)OCCOc2ccc3ccc(=O)oc3c2)cc1]
2′-(2-Oxo-2H-chromen-7-yloxy)ethyl 4″-methylbenzenesulfonate; 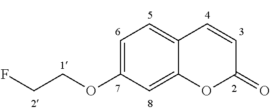 [SMILES: O=c1ccc2ccc(OCCF)cc2o1]
7-(2′-Fluoroethoxy)-2H-chromen-2-one; 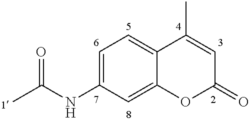 [SMILES: CC(=O)Nc1ccc2c(C)cc(=O)oc2c1]
N-(4-Methyl-2-oxo-2H-chromen-7-yl)acetamide; 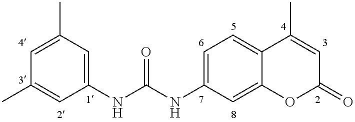 [SMILES: Cc1cc(C)cc(NC(=O)Nc2ccc3c(C)cc(=O)oc3c2)c1]
1-(3′,5′-dimethylphenyl)-3-(4-methyl-2-oxo-2H-chromen-7-yl)urea; 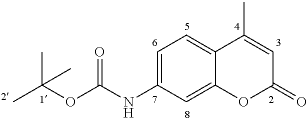 [SMILES: Cc1cc(=O)oc2cc(NC(=O)OC(C)(C)C)ccc12]
tert-Butyl 4-methyl-2-oxo-2H-chromen-7-ylcarbamate;

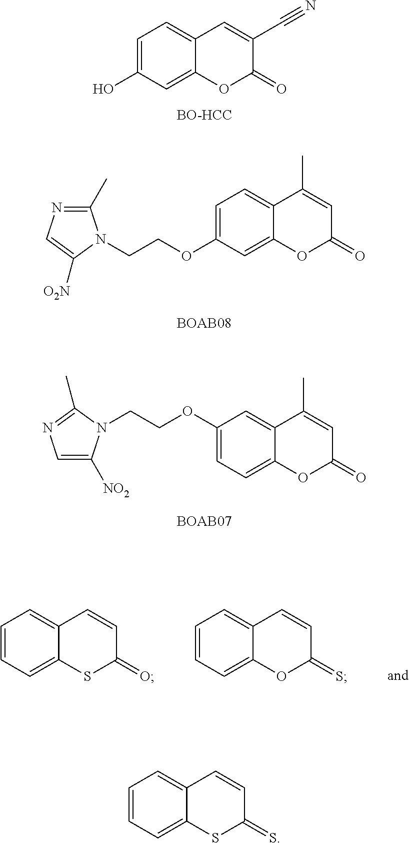

37. The method of claim 31, further comprising treating the mammal with additional anticancer agents.

38. The method of claim 36, wherein the mammal is also treated with additional anticancer agents.

39. The method of claim 31 wherein the mammal has breast cancer, lung carcinoma, pancreatic carcinoma, renal carcinoma, ovarian, prostate or cervical carcinoma, glioblastoma, colorectal carcinoma.

40. The method of claim 31 wherein the mammal is a human.

41. The method of claim 36 wherein the mammal has breast cancer, lung carcinoma, pancreatic carcinoma, renal carcinoma, ovarian, prostate or cervical carcinoma, glioblastoma, colorectal carcinoma.

42. The method of claim 36 wherein the mammal is a human.

43. A compound selected from the following group:
2′,4′,6′-trimethyl-1-(2-(2-oxo-2H-chromen-7-yloxy)ethyl)pyridine;
7-(pent-4-ynyloxy)-2H-chromen-2-one hexacarbonyldicobalt;
7-(prop-2-ynyloxy)-2H-chromene-2-thione hexacarbonyldicobalt;
7-(prop-2-ynyloxy)-2H-chromen-2-one hexacarbonyldicobalt;
N-(4-methyl-2-oxo-2H-chromen-7-yl)-4′-methylbenezenesulfonimde;
7-(prop-2-ynyloxy)-2H-chromene-2-thione;
7-(allyloxy)-2H-chromene-2-thione;
2-(2-oxo-2H-chromen-7-yloxy)ethylcarbamate;
1-(4-(4-((2-oxo-2H-chromen-6-yloxy)methyl)-1H-1,2,3-triazol-1-yl)-tetrahydro-5-(hydroxymethyl)furan-2-yl)-5-methylpyrimidine-2,4(1H,3H)dione;
7-[(1′H-1′,2′,3′-triazol-4′-yl)methoxy]-2H-chromen-2-one;
6-((1-(2-chlorophenyl)-1H-1,2,3-triazol-4-yl)methoxy)-2H-chromen-2-one
6-((1-(2-bromophenyl)-1H-1,2,3-triazol-4-yl)methoxy)-2H-5-chromen-2-one;
6-((1-(2-fluorophenyl)-1H-1,2,3-triazol-4-yl)methoxy)-2H-5-chromen-2-one;
6-((1-(2-iodophenyl)-1H-1,2,3-triazol-4-yl)methoxy)-2H-5-chromen-2-one;
7-(2′,4′,6′-trimethylpyridinium)-4-methyl-2H-chromen-2-one;
7-(2′,4′,6′-trimethylpyridinium)-4-methyl-2H-chromen-2-one perchlorate;
4-methyl-7-(4-((2-oxo-2H-chromen-7-yloxy)methyl)-1H-1,2,3-triazol-1-yl)-2H-chromen-2-one; 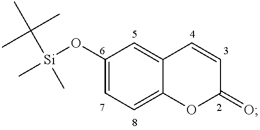 [SMILES: CC(C)(C)[Si](C)(C)Oc1ccc2oc(=O)ccc2c1]
6-(tert-Butyldimethylsilyloxy)-2H-chromen-2-one; 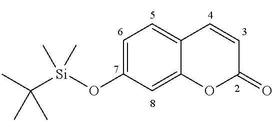 [SMILES: CC(C)(C)[Si](C)(C)Oc1ccc2ccc(=O)oc2c1]
7-(tert-Butyldimethylsilyloxy)-2H-chromen-2-one; 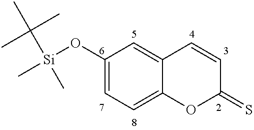 [SMILES: CC(C)(C)[Si](C)(C)Oc1ccc2oc(=S)ccc2c1]
6-(tert-Butyldimethylsilyloxy)-2H-chromene-2-thione; 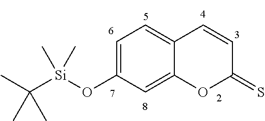 [SMILES: CC(C)(C)[Si](C)(C)Oc1ccc2ccc(=S)oc2c1]
7-(tert-Butyldimethylsilyloxy)-2H-chromene-2-thione; 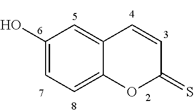 [SMILES: Oc1ccc2oc(=S)ccc2c1]
6-Hydroxy-2H-chromene-2-thione; 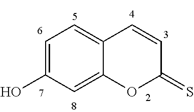 [SMILES: Oc1ccc2ccc(=S)oc2c1]
6-Hydroxy-2H-chromene-2-thione; 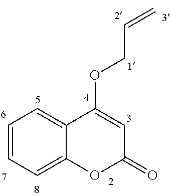
4-(Allyloxy)-2H-chromen-2-one; 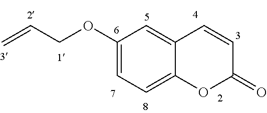
6-(Allyloxy)-2H-chromen-2-one; 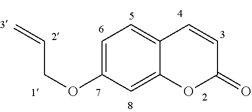
7-(Allyloxy)-2H-chromen-2-one; 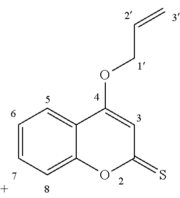
4-(Allyloxy)-2H-chromene-2-thione; 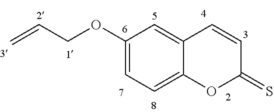
6-(Allyloxy)-2H-chromene-2-thione; 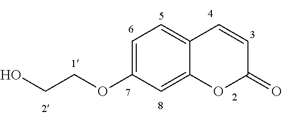 [SMILES: O=c1ccc2ccc(OCCO)cc2o1]
7-(2′-hydroxyethoxy)-2H-chromen-2-one; 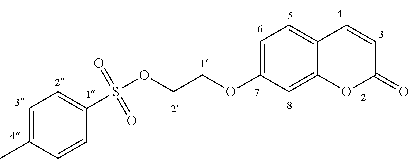 [SMILES: Cc1ccc(S(=O)(=O)OCCOc2ccc3ccc(=O)oc3c2)cc1]
2′-(2-Oxo-2H-chromen-7-yloxy)ethyl 4″-methylbenzenesulfonate; 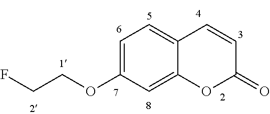 [SMILES: O=c1ccc2ccc(OCCF)cc2o1]
7-(2′-Fluoroethoxy)-2H-chromen-2-one; 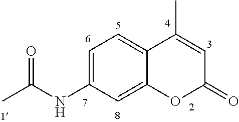 [SMILES: CC(=O)Nc1ccc2c(C)cc(=O)oc2c1]
N-(4-Methyl-2-oxo-2H-chromen-7-yl)acetamide; 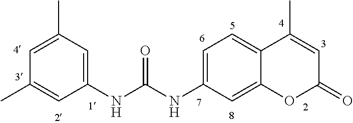 [SMILES: Cc1cc(C)cc(NC(=O)Nc2ccc3c(C)cc(=O)oc3c2)c1]
1-(3′,5′-dimethylphenyl)-3-(4-methyl-2-oxo-2H-chromen-7-yl)urea; 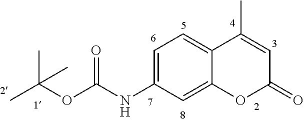 [SMILES: Cc1cc(=O)oc2cc(NC(=O)OC(C)(C)C)ccc12]
tert-Butyl 4-methyl-2-oxo-2H-chromen-7-ylcarbamate;

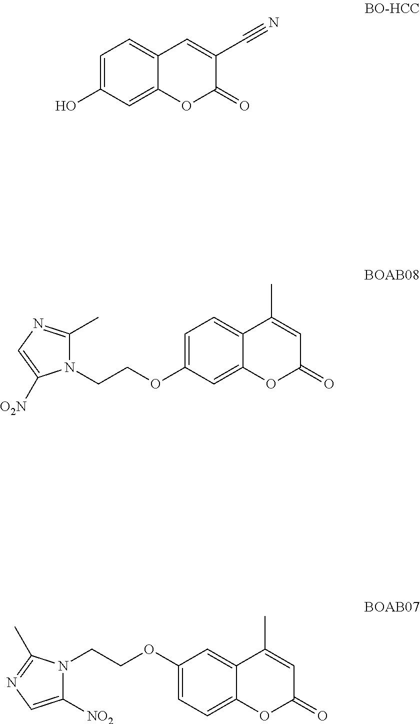 [SMILES: N#Cc1cc2ccc(O)cc2oc1=O ; Cc1cc(=O)oc2cc(OCCn3c([N+](=O)[O-])cnc3C)ccc12 ; Cc1cc(=O)oc2ccc(OCCn3c([N+](=O)[O-])cnc3C)cc12]

44. A pharmaceutical composition comprising a compound of claim 43 or a salt thereof and a pharmaceutically acceptable excipient.
